(19) United States
(12) Patent Application Publication
(10) Pub. No.: US 2019/0352663 A1
(43) Pub. Date: Nov. 21, 2019

(54) PLANTS RESISTANT TO HUANGLONGBING

(71) Applicants: The Regents of the University of California, Oakland, CA (US); The University of Florida Research Foundation, Inc., Gainesville, FL (US)

(72) Inventors: Wenbo MA, Riverside, CA (US); Gitta COAKER, Davis, CA (US); Nian WANG, Gainesville, FL (US)

(21) Appl. No.: 16/399,660

(22) Filed: Apr. 30, 2019

Related U.S. Application Data
(60) Provisional application No. 62/664,847, filed on Apr. 30, 2018.

(51) Int. Cl. C12N 15/82; C12N 9/22

(52) U.S. Cl. CPC C12N 15/8281; A01H 6/785; C12N 9/22

(57) ABSTRACT

Provided herein are genetically modified citrus plants having enhanced resistance to Huanglongbing (HLB) and methods of producing such plants by overexpressing a papain-like cysteine protease (PLCP) polypeptide to overcome the function of Sec-delivered effector 1 (SDE1).

### CROSS-REFERENCES TO RELATED APPLICATIONS

This application claims priority to U.S. Provisional Application No. 62/664,847, filed Apr. 30, 2018, the entire content of which is incorporated by reference herein for all purposes.

### STATEMENT AS TO RIGHTS TO INVENTIONS MADE UNDER FEDERALLY SPONSORED RESEARCH AND DEVELOPMENT

This invention was made with Government support under U.S. Department of Agriculture National Institutes of Food and Agriculture (USDA-NIFA) Grant No. 2016-70016-24833. The government has certain rights in this invention.

### REFERENCE TO SUBMISSION OF A SEQUENCE LISTING AS A TEXT FILE

The instant application contains a Sequence Listing which has been filed electronically in ASCII format and is hereby incorporated by reference in its entirety. Said ASCII copy, created on Jul. 16, 2019, is named 081906-1137135_229210US_SL.txt and is 20,306 bytes in size.

### BACKGROUND OF THE INVENTION

Huanglongbing (HLB), or citrus greening disease, is currently considered the most destructive disease of citrus worldwide^{1,2,3,4,5}. In the major citrus-growing areas including the US and Asia, the presumed causal agent of HLB is a gram-negative bacterium, Candidatus Liberibacter asiaticus (CLas). CLas is transmitted to citrus by the Asian citrus psyllid (ACP) during sap feeding, where it then colonizes the phloem sieve elements, eventually leading to disease symptoms. Infected trees exhibit leaf mottling, deformed/discolored fruits, premature fruit drop, and premature mortality2. In the US, Florida has lost over $7 billion in total industry output due to HLB since it was first detected in 2005 till 2014^{6,7}. Secreted proteins of pathogens, called effectors, play an essential role in bacterial pathogenesis. Collectively, effectors aid infection by suppressing plant immunity and creating environments favorable for colonization and proliferation^{8,9}. Many gram-negative bacteria ‘inject’ effectors directly into host cells through the type III secretion system¹⁰. In contrast, insect-transmitted bacteria, like CLas, often lack this specialized delivery machinery, but can utilize the general Sec secretion system to release effectors¹¹. These Sec-delivered effectors (SDEs) carry an N-terminal secretion signal, allowing their export from pathogen cells into the extracellular space. The essential roles of SDEs in bacterial virulence are best illustrated by insect-transmitted, phloem-colonizing phytoplasmas, where expression of their individual SDEs in Arabidopsis thaliana leads to phenotypes that mimic disease symptoms^{12,13}. Sequence analysis of the CLas genome revealed that it encodes all the components of the Sec secretion machinery¹⁴. In addition, 86 proteins were confirmed to possess a functional Sec-secretion signal, indicating that they could potentially be released by CLas into the phloem during infection¹⁵. A few of these SDEs exhibited higher expression levels in citrus relative to their levels of expression in ACP^{14,15}, indicating that they may contribute to CLas colonization and/or disease progression in citrus. However, our knowledge on the cellular function of CLas SDEs in plant or insect hosts is lacking.

### BRIEF SUMMARY OF THE INVENTION

In some embodiments, plant or plant cell (e.g., a citrus plant or plant cell) comprising a papain-like cysteine protease (PLCP) polypeptide is provided, wherein the PLCP polypeptide is heterologously expressed or is a mutant PLCP that has reduced binding to Candidatus Liberibacter asiaticus (CLas) effector SDE1 compared to a corresponding wildtype PLCP protein.

In some embodiments, the PLCP polypeptide (which can be a native or mutated version thereof) is heterologously expressed. In some embodiments, the citrus plant is a transgenic citrus plant comprising a heterologous expression cassette comprising a promoter operably linked to a nucleic acid sequence encoding the PLCP polypeptide. In some embodiments, the PLCP is encoded from an endogenous coding sequence that is linked to a modified PLCP promoter sequence comprising at least one (e.g., 1, 1-2, 1-5, 1-10, 1-20) nucleotide alteration compared to the native PLCP promoter sequence.

In some embodiments, the PLCP polypeptide is a mutant PLCP that has reduced binding to Candidatus Liberibacter asiaticus (CLas) effector SDE1 compared to a corresponding wildtype PLCP protein, wherein the mutant PLCP has between 1-10 (e.g., 1, 2, 3, 4, 5, 6, 7, 8, or 9) amino acid changes compared to the wildtype PLCP protein.

In some embodiments, the citrus plant has enhanced resistance to infection or damage by CLas compared to a control plant (e.g., otherwise identical) that lacks the heterologously expressed PLCP polypeptide or mutant PLCP. In some embodiments, the PLCP polypeptide is from a PLCP subfamily of SAG12, THL1, CEP1, XCP1, XBCP3, RD21a, RD19, AALP and CTB. In some embodiments, the PLCP polypeptide is substantially (e.g., at least 70, 80, 85, 90, 95, 98, 99%) identical to a PLCP polypeptide encoded by a gene listed in FIG. 1 or otherwise described herein. In some embodiments, the PLCP polypeptide comprises a mature region that is substantially (e.g., at least 70, 80, 85, 90, 95, 98, 99%) identical to the mature region of a PLCP polypeptide listed in FIG. 1 or otherwise described herein. In some embodiments, the PLCP polypeptide has at least 70% identity, or at least 75%, 80%, 85%, 90%, or 95% identity, to a native SAG12-1, SAG12-3, or RD19 polypeptide sequence. In some embodiments, the PLCP polypeptide comprises a mature region that has at least 70% identity, or at least 75%, 80%, 85%, 90%, or 95% identity, to the mature region of a native SAG12-1, SAG12-3, or RD19 polypeptide sequence. In some embodiments, the PLCP polypeptide has at least 70% identity, or at least 75%, 80%, 85%, 90%, or 95% identity, to the amino acid sequence of SEQ ID NO:1 or SEQ ID NO:2. In some embodiments, the PLCP polypeptide comprises a mature region that has at least 70% identity, or at least 75%, 80%, 85%, 90%, or 95% identity, to amino acids 21-344 of SEQ ID NO:1 or amino acids 36-348 of SEQ ID NO:2. In some embodiments, the polynucleotide encoding a PLCP polypeptide for heterologous expression encodes the mature region of a PLCP polypeptide as described herein, operably linked to a heterologous signal peptide.

Also provided is a method of making citrus plant has enhanced resistance to infection or damage by CLas. In some embodiments, the method comprises introducing into a citrus plant an alteration in a promoter operably linked to a nucleic acid sequence encoding a native papain-like cysteine protease (PLCP) polypeptide, wherein the alteration results in increased expression the PLCP polypeptide compared to the native PLCP polypeptide.

In some embodiments, the promoter is a native promoter and the introducing comprises introducing a targeted nuclease that cleaves a target region in that native promoter and a heterologous nucleic acid sequence is introduced into the native promoter thereby introducing the alteration into the native promoter. In some embodiments, the nuclease is an RNA-guided nuclease. In some embodiments, the RNA-guided nuclease is a Cpf1 nuclease or a Cas9 nuclease and the method further comprises introducing into the cell a guide RNA that specifically hybridizes to the target region.

Also provided is a nucleic acid expression cassette comprising a promoter operably linked to a polynucleotide encoding the mutant PLCP wherein the mutant PLCP has between 1-10 (e.g., 1, 2, 3, 4, 5, 6, 7, 8, or 9) amino acid changes compared to a (e.g., otherwise identical) wildtype PLCP protein.

### BRIEF DESCRIPTION OF THE DRAWINGS

FIG. 1 SDE1 interacts with citrus papain-like cysteine proteases. Panel a, Yeast-two-hybrid (Y2H) assays using the CLas effector SDE1 as the bait and full-length citrus papain-like cysteine proteases (CsPLCPs), representing different subfamilies as the prey. SDE1 was cloned into the vector pGBKT7 and individual CsPLCPs were cloned into the vector pGADT7. Growth of yeast cells on SD-3 selective media represents protein-protein interaction, growth of the same cells on SD-2 media confirms yeast transformation. Yeast transformed with the empty vectors served as negative controls. The initial PLCP found from Y2H screening (CsSAG12-1, XM_006495158) is indicated with an asterisk (*). The gene IDs of the other interacting PLCPs are CsSAG12-2 (XM_006470229), CsXCBP3 (orange1.1g012960), CsRD21a (XM_006473212), CsRD19 (orange1.1g017548), CsAALP (XM_006474664), and CsCTB (orange1.1g018568). Panel b, Phylogeny and subfamily classification of canonical PLCPs in the C. sinensis (sweet orange) genome. The phylogenetic tree was made with MEGA6.06 (100 bootstrap replicates, Maximum Likelihood method, Jones-Taylor-Thornton model), using the Arabidopsis thaliana PLCP subfamily classification22. The asterisk (*) indicates the initially found CsSAG12-1. Panel c, Y2H assay examining the interaction of SDE1 with the cysteine protease domain of CsPLCPs. Panel d, In vitro pull-down assay using the GST-tagged cysteine protease domain of CsPLCPs to immunoprecipitate SDE1 protein. Input and immunoprecipitated proteins (output) were visualized by western blotting using anti-GST and anti-SDE1 antibodies. Asterisks (*) indicate the protein bands that correspond to individual CsPLCPs. GST-tagged Arabidopsis Double-stranded DNA binding protein 4 (AtDRB4) was used as a negative control.

FIG. 2 SDE1 inhibits PLCP activity in vitro and in plant cells: Panel a, Proteolytic activity of papain measured by digestion of a fluorescent casein substrate in the presence of E-64, purified SDE1 protein, or BSA (as a negative control). Fluorescence was measured at 485/530 nm excitation/emission. Mean ±standard deviation (n=3) is shown. Asterisks (*) indicate statistically significant differences based on the two-tailed Student's t-test. p<0.01=**, p<0.001=***. Panel b, Inhibitory effect of SDE1 on the protease activity of papain examined by activity-based protein profiling (ABPP). Active papain was labeled by DCG-04 in the presence of 10 μM E-64 or 1.6 μM purified SDE1 protein and detected using streptavidin conjugated with horseradish peroxidase (HRP). Panel c, SDE1 inhibits the activity of CsRD21a. CsRD21a-Flag (with its N-terminal secretion signal) was expressed in N. benthamiana. Active protease in the apoplastic fluid was labeled via ABPP. ImageJ analysis of the signal intensity revealed approximately 9%, 62%, and 96% reduction of CsRD21a activity in the presence of 0.8, 1.6, or 3.2 μM purified SDE1 protein, respectively. Panel d, SDE1 inhibits PLCP activity in citrus. Total protein extracts from Navel orange (C. sinensis) leaves were labeled via ABPP in the presence of 120 nM purified SDE1 protein. Active proteases were enriched using streptavidin beads and detected using streptavidin-HRP conjugates. Panel e, Transgenic grapefruit (Duncan) seedlings expressing SDE1 exhibit reduced protease activity. Five individual lines were analyzed by ABPP. SDE1-10 does not have significant SDE1 protein accumulation and served as a negative control.

FIG. 3 CsPLCPs accumulate during SA treatment and infection: Panel a, Abundance of PLCP genes was determined by quantitative RT-PCR after SA treatment. One-year-old Navel oranges (C. sinensis) were sprayed with 2 mM SA or water. Leaf samples were collected after 48 hours for RNA extraction and PCR analyses. Cytochrome oxidase subunit 1 (COX, KF933043.1) was used as the internal standard. Graph shows mean±standard error of three replicates. Asterisks (*) indicate statistically significant differences based on the two-tailed Student's t-test. p<0.05=*,p<0.01=**. Panel b, Protein abundance of PLCPs was determined in healthy (−) or CLas-infected (+) citrus branches using an anti-AALP antibody. Freshly cut stems were stamped onto nitrocellulose membranes and PLCPs and SDE1 were detected using western blotting. The titer of CLas in each sample was evaluated by quantitative PCR with observed Ct values of 27.97 for symptomatic tissue (+S), and not detected for asymptomatic tissue from the same infected tree (+AS) or tissue from an uninfected tree (−). Ponceau-stained membrane was shown as a control.

FIG. 4 CsSAG12s increase in abundance but not activity in infected citrus: Panel a, Diagram illustrating the experimental approach for detecting abundance and activity of CsPLCPs in healthy and infected Navel oranges (C. sinensis) from a Texas grove using mass spectrometry. Panel b, Abundance and protease activity of six PLCPs belonging to four subfamilies in citrus leaf samples. Leaf tissue was ground in Tris buffer and divided into two to assess abundance and activity. PLCP abundance was tested using an in-solution digest coupled with mass spectrometry. For activity, the addition of DCG-04 permits the labeling of active PLCPs. Active PLCPs were captured and identified by streptavidin IP coupled with mass spectrometry. Mean±standard error of three replicates is shown. Asterisks (*) indicate statistically significant differences based on the two-tailed Student's t-test. N.d=no difference, p<0.05=*,p<0.01=**,p<0.001=***. The gene IDs are CsXBCP3 (orange1.1g012960m), CcXCP1 (Ciclev10001665m), CsAALP (orange1.1g036910m), CcSAG12-1 (Ciclev10005334m), CsSAG12-3 (orange1.1g018958m), and CsSAG12-4 (orange1.1g019063m).

FIG. 5 SDE1 promotes Pseudomonas syringae infection of Arabidopsis: Mature leaves of five-week old plants of Arabidopsis thaliana ecotype Col-0 were syringe-infiltrated with cell suspensions of P. syringae pv. tomato (Pto) strains including DC3000 (wild-type), Δcip1, Δcip1(empty vector, EV), and Δcip1(SDE1). Bacterial titers were determined as colony forming units (cfu/cm²) at the time of infiltration (Day 0) and three days post infiltration (Day 3). Graph shows mean±standard deviation of data from three independent experiments. Different letters (a, b, and c) indicate statistically significant differences (p<0.05) based on a one-way ANOVA followed by Tukey's HSD post hoc test.

FIG. 6 SDE1 does not inhibit Solanaceous RCR3 activity: Panel a, SDE1 did not elicit cell death in tomato (Moneymaker, Solanum lycopersicum) leaves expressing the immune receptor Cf-2. Purified recombinant proteins FLAG-Avr2-6XHIS (“6XHIS” disclosed as SEQ ID NO: 3) or FLAG-6XHIS-SDE1 (“6XHIS” disclosed as SEQ ID NO: 3) were infiltrated in tomato leaves in three different concentrations: 1 μg, 100 ng, and 10 ng. Avr2 inhibits the protease activity of RCR3^{pim} (RCR3 from Solanum pimpinellifolium, which is required for Avr2-triggered cell death in tomato plants expressing Cf-2 produced by S. pimpinellifolium (left). Cf-0 (right) and Cf-2 rcr3 (middle, containing a rcr3 mutant with a premature stop codon) tomatoes were used as negative controls. Picture was taken after 7 dpi. Panel b, SDE1 does not inhibit the activity of RCR3^{pim}. Full-length of RCR3^{pim}-HIS was transiently expressed in N. benthamiana and secreted into the apoplast. Apoplastic fluid was extracted and the active protease was labeled via ABPP in the presence of 0.8, 1.6 or 3.2 μM of purified SDE1 protein. Coomassie brilliant blue (CBB) served as a loading control.

FIG. 7 PLCP domain architecture and structural mode of SAG12-1 from Citrus sinensis. Panel a, Canonical PLCPs possess a signal peptide (SP), an autoinhibitory pro-peptide, and the catalytic protease domain. The predicted catalytic triad of papain (cysteine159, histidine293 and asparagine309) are presented as an example. Panel b, To generate a structural model for CsSAG12-1, the predicted protease domain was submitted to MODELLER, revealing 56% sequence similarity to the CysEP PLCP from Ricinus communis (PDB: 1S4V). PDB 1S4V was used as a template for structural modeling of CsSAG12-1 and visualized in CHIMERA.

FIG. 8 SDE1 interacts with additional PLCPs from the SAG12 subfamily. Pairwise yeast-two-hybrid (Y2H) assay using SDE1 as bait and the cysteine protease domains of PLCPs (Cs SAG12-1, CsSAG12-3, CcSAG12-4) as the prey. Growth of yeast on SD-4 selective medium represents protein-protein interaction, growth of yeast on SD-2 medium confirms yeast transformation. Yeast cells transformed with pGBKT7 and pGADT7 empty vectors severed as negative controls. CsAALP (full-length) and CsAALP-Cys (protease domain only) served as positive controls.

FIG. 9 DE1 but not SDE2 can inhibit the protease activity of papain. Proteolytic activity of papain measured by digestion of fluorescent casein substrates in the presence of 1 μM E-64, purified SDE1 (0.74 and 0.15 μM) or SDE2 (0.3 μM) protein (CLIBASIA_03230), and BSA (0.74 and 0.15 μM). Fluorescence was measured at 485 nm excitation over 530 nm emission. Values are average of duplicates with the Standard Deviation shown as the error bars. Statistical analysis was done using Student's two-tailed t-test and significant differences are labeled with asterisks. p<0.05=*

FIG. 10 E-64 inhibits the interaction between SDE1 and PLCPs. In vitro pull-down assay using the GST-tagged cysteine protease domain of CsPLCPs to immunoprecipitate SDE1 in the presence or absence of E-64. Input and immunoprecipitated proteins (output) were visualized by western blotting using anti-GST and anti-SDE1 antibodies. Asterisks (*) indicate the protein bands that correspond to individual PLCPs. In addition to two PLCPs from citrus (CsRD21a and CsSAG12-1), RCR3 from tomato (SdRCR3) was also examined. Since E-64 binds the catalytic site of PLCPs, inhibition of E-64 on SDE1/PLCP interaction indicates SDE1 binds to PLCPs at or around the catalytic sites.

FIG. 11 SDE1 proteins accumulate in transgenic citrus seedlings. Leaves from individual transgenic citrus lines (one-year old seedlings) were ground with liquid nitrogen into powder and re-suspended in 2× Laemmli loading dye. Samples were boiled for 5 min, then separated on a 12% protein gel for western blotting. SDE1 proteins were detected by anti-HA antibody (Santa Cruz Biotechnology, CA). Gel stained with Coomassie brilliant blue (CBB) served as a loading control. Leaf tissue from wild-type (WT) grapefruit seedlings of the same age were included as controls.

FIG. 12 The anti-AALP antibody specifically recognizes citrus PLCP(s). Two hundred fifty micrograms of citrus leaf extract was incubated with and without E-64 for 30 min prior to the addition of DCG-04. Active PLCPs were captured by streptavidin IP coupled with anti-streptavidin western blotting or anti-AALP western blotting. Coomassie brilliant blue (CBB) stained gel served as a loading control.

FIG. 13 SDE1 is expressed and secreted by Pseudomonas syringae in an inducible medium. SDE1-HA (full-length gene including sequences corresponding to the N-terminal secretion signal) was cloned in the plasmid vector pUCP20tk containing the promoter of hopZ1a. Empty vector and pUCP20tk::SDE1 were introduced into P. syringae pv. tomato DC3000 Δcip1 knockout mutant by electroporation. Transformants were grown in the M63 minimal medium (pH 5.3) to induce SDE1 expression. The secretion of SDE1 proteins was detected in the supernatant of the cell culture using western blotting. Coomassie brilliant blue (CBB) stained gel served as a loading control.

FIG. 14 SDE1 does not inhibit the activity of the wild potato RCR3^{dms3}. Full-length of RCR3 from the wild potato species Solanum demissum (RCR3^{dms3}) was transiently expressed in N. benthamiana and secreted into the apoplast. Apoplast fluid was extracted and the active protease was labeled using DCG-04 in the presence of 0.8, 1.6 or 3.2 μM of purified SDE1 protein. Coomassie brilliant blue (CBB) served as a loading control.

FIG. 15 Supplementation of papain in culture media does not inhibit the growth of PtoDC3000Δcip1. PtoDC3000Δcip1 containing pUCP20tk empty vector was grown in Panel a, King's B medium or Panel b, hrp-inducing minimal medium in the presence of either 100 μg/mL papain (black diamonds) or 100 μg/mL BSA (grey circles) and the optical density of the cultures (OD₆₀₀) was monitored over time. Graphs show mean±standard deviation of three replicates. This experiment was repeated once with similar results.

FIG. 16 A potential model of SDE1 and PLCP interaction in CLas-infected citrus. After infection, CLas proliferates in phloem sieve elements. Sieve elements are dependent upon adjacent, metabolically active companion cells. Citrus is able to perceive the bacterial pathogen and induce defense responses, including increased PLCP accumulation. These proteins might be secreted into the sieve elements. CLas possesses the Sec secretion system and secretes multiple Sec-delivered effectors, including SDE1, which acts to inhibit the protease activity of PLCPs. SDE1 can move through the sieve elements and might be able to translocate into adjacent companion cells to suppress this PLCP-based defense responses and promote bacterial infection.

FIG. 17 provides illustrative data showing expression patterns of PLCP genes in seven citrus varieties, including HLB-tolerant varieties (Sugar Belle, Australian finger lime, and Carrizo) and susceptible varieties (Clementine, Sweet oranges, Pumelo and Alemow) by Nanostring.

### Definitions

As used herein, the terms “citrus greening disease” and “Huanglongbing (HLB)” refer to a bacterial infection of plants (e.g., citrus plants) caused by bacteria in the genus Candidatus Liberibacter (Candidatus Liberibacter asiaticus, Candidatus Liberibacter africanus, and Candidatus Liberibacter americanus). The infection is vectored and transmitted by the Asian citrus psyllid, Diaphorina citri, and the African citrus psyllid, Trioza erytreae. Three different types of HLB are currently known: the heat-tolerant Asian form, and the heat-sensitive African and American forms.

As used herein, the term “HLB resistance” refers to the ability of a plant to not be affected by HLB or infection by Candidatus Liberibacter bacteria or to have reduced symptoms compared to a control counterpart (e.g., wildtype citrus plant or a plant of the same genetic background that does not have a genetic modification as described herein to enhance HLB resistance) infected by HLB under the same conditions.

As used herein, the term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same.

In any of the compositions or methods described in the present disclosure, any plant species can be used. In some embodiments, the plant species is from the genus Citrus (e.g., Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus sinensis, and Citrus tangerina).

In some embodiments, the plant can be selected from the group consisting of Citrus reticulata, Citrus sinensis, Citrus clementina, Capsicum annuum, Solanum tuberosum, Solanum lycopersicum, Solanum melongena, and Nitotiana benthamiana. In particular embodiments, the plant is a sweet orange plant (Citrus sinensis). In some embodiments, the plant can be a clementine plant (Citrus clementina).

As used herein, the term “nucleic acid” or “polynucleotide” refers to a single or double-stranded polymer of deoxyribonucleotide or ribonucleotide bases read from the 5′ to the 3′ end. Nucleic acids may also include modified nucleotides that permit correct read through by a polymerase and do not significantly alter expression of a polypeptide encoded by that nucleic acid.

As used herein, the terms “peptide” and “polypeptide” are used interchangeably and describe a single polymer in which the monomers are amino acid residues which are joined together through amide bonds. A polypeptide is intended to encompass any amino acid sequence, either naturally occurring, recombinant, or synthetically produced.

As used herein, the phrase “nucleic acid encoding” or “polynucleotide encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full-length sequences derived from the full-length sequences. It should be further understood that the sequence includes the degenerate codons of the native sequence or sequences which may be introduced to provide codon preference in a specific host cell.

Two nucleic acid sequences or polypeptides are said to be “identical” if the sequence of nucleotides or amino acid residues, respectively, in the two sequences is the same when aligned for maximum correspondence as described below. “Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. When percentage of sequence identity is used in reference to proteins or peptides, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller⁷⁷, e.g., as implemented in the program PC/GENE (Intelligenetics, Mountain View, Calif., USA).

As used herein, the term “substantial identity” or “substantially identical,” as used in the context of polynucleotide or polypeptide sequences, refers to a sequence that has at least 50% sequence identity to a reference sequence. Alternatively, percent identity can be any integer from 50% to 100%. Exemplary embodiments include at least: 50%, 55%, 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. These values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually 5 about 50 to about 200, more usually about 100 to about 150 in which a sequence may be compared to a reference sequence of the same number of contiguous positions after the two sequences are optimally aligned. Methods of alignment of sequences for comparison are well-known in the art.

Algorithms that are suitable for determining percent sequence identity and sequence similarity are the BLAST and BLAST 2.0 algorithms, which are described in Altschul et al. (1990)⁸¹ and Altschul et al. (1977)⁸², respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold^{81,82}. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAS TN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff⁸³). For purposes of this application, amino acid sequence identity is determined using BLASTP with default parameters.

The BLAST algorithm also performs a statistical analysis of the similarity between two sequences (see, e.g., Karlin & Altschul⁸⁴). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “complementary to” is used herein to mean that a polynucleotide sequence is complementary to all or a portion of a reference polynucleotide sequence. In some embodiments, a polynucleotide sequence is complementary to at least 15, at least 20, at least 25, at least 30, at least 40, at least 50, at least 75, at least 100, at least 125, at least 150, at least 175, at least 200, or more contiguous nucleotides of a reference polynucleotide sequence. In some embodiments, a polynucleotide sequence is “substantially complementary” to a reference polynucleotide sequence if at least 70%, at least 75%, at least 80%, at least 85%, at least 90%, or at least 95% of the polynucleotide sequence is complementary to the reference polynucleotide sequence.

As used herein, a polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

As used herein, an “expression cassette” refers to a nucleic acid construct, which when introduced into a host cell, results in transcription and/or translation of a RNA or polypeptide, respectively. Antisense constructs or sense constructs that are not or cannot be translated are expressly included by this definition. The inserted polynucleotide sequence need not be identical but may be only substantially similar to a sequence of the gene from which it was derived.

As used herein, the term “host cell” refers to any cell capable of replicating and/or transcribing and/or translating a heterologous polynucleotide. Thus, a “host cell” refers to any prokaryotic cell (including but not limited to E. coli) or eukaryotic cell (including but not limited to yeast cells, mammalian cells, avian cells, amphibian cells, plant cells, fish cells, and insect ceils), whether located in vitro or in vivo. For example, host cells may be located in a transgenic animal or transgenic plant, prokaryotic cells (including but not limited to E. coli) or eukaryotic cells (including but not limited to yeast cells, mammalian cells, avian cells, amphibian cells, plant cells, fish cells, and insect cells). Host cells can be for example, transformed with the heterologous polynucleotide.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing, tissue, and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

### DETAILED DESCRIPTION OF THE INVENTION

The citrus industry is facing an unprecedented challenge from Huanglongbing (HLB). The devastating impact of HLB on the citrus industry warrants immediate yet sustainable solutions, which we are only beginning to unveil. All citrus cultivars can be affected by the HLB-associated bacterium Candidatus Liberibacter asiaticus (CLas) and there is no known resistance. Advances in understanding of the molecular interactions between CLas and citrus will provide the fundamental knowledge needed to develop robust HLB management techniques. As shown herein, we used the effector Sec-delivered effector 1 (SDE1) as a molecular probe to reveal PLCPs as virulence targets of CLas in citrus, thereby providing one of the first mechanistic insights into HLB pathogenesis.

SDE1, which is conserved in all CLas isolates, was used as a molecular probe to understand CLas virulence. While not wanting to be limited by theory, it was discovered that SDE1 directly interacts with citrus papain-like cysteine proteases (PLCPs) and inhibits protease activity. PLCPs are defense-inducible and exhibit increased protein accumulation in CLas-infected trees, suggesting a role in citrus defense responses. PLCP activity in field samples was analyzed, revealing specific members that increase in abundance but remain unchanged in activity during infection. SDE1-expressing transgenic citrus also exhibited reduced PLCP activity. These data demonstrate that SDE1 inhibits citrus PLCPs, which are immune-related proteases that enhance defense responses in plants.

The CLas effector SDE1 (CLIBASIA_05315) was characterized and its targets identified in citrus. SDE1 is conserved across CLas isolates with a typical Sec-dependent secretion signal^{15,16,17}. The expression of SDE1 is ˜10-fold higher in citrus than in ACP¹⁶, indicating a role in CLas colonization of plant hosts. SDE1 is also highly expressed in asymptomatic tissues, suggesting a potential virulence function during early infection stages. It was found that SDE1 interacts with multiple members of papain-like cysteine proteases (PLCPs), which are known to regulate defense in Arabidopsis and solanaceous crops against bacterial, fungal, and oomycete pathogens^{18,19}. The abundance of PLCPs is increased in citrus infected with CLas, likely as a defense response. SDE1 can directly inhibit PLCP activity in vitro and in citrus. Using a surrogate system, it was further shown that SDE1 was able to promote bacterial infection in Arabidopsis. Taken together, this research advances our understanding of HLB pathogenesis by identifying citrus targets of a conserved CLas effector, which could be exploited for HLB management.

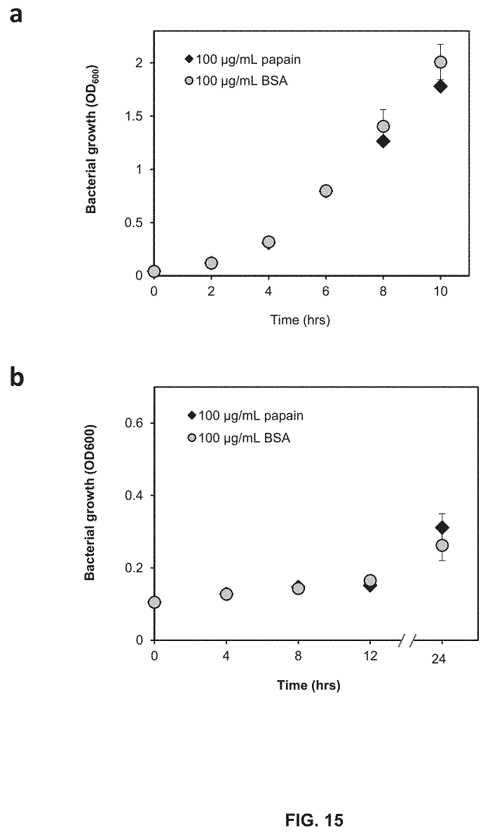

PLCPs have been reported to regulate plant immunity and contribute to defense against a broad range of pathogens including bacteria, fungi, and oomycetes^{20,21,25,32}. For example, the SAG12 subfamily members, RCR3 and PIP1, in tomato contribute to defense against the oomycete pathogen Phytophthora infestans^{25,34}. Knocking out or silencing specific PLCP genes in Arabidopsis, tomato, and N. benthamiana resulted in increased susceptibly to various pathogens^{19,35}. The mechanisms underlying PLCP-mediated defense could work on multiple levels. They may directly hydrolyze pathogen components—for example, growth inhibition by papain against the papaya pathogen Phytophthora palmivora was recently reported³⁶. However, an inhibitory effect of papain on bacterial growth in artificial media was not observed (FIG. 15), suggesting that direct antimicrobial activity by PLCPs is highly specific. It is possible that PLCPs contribute to the citrus response to CLas by regulating defense signaling. For example, it was proposed that PLCPs could cleave microbial or host peptides to elicit defense responses¹⁹.

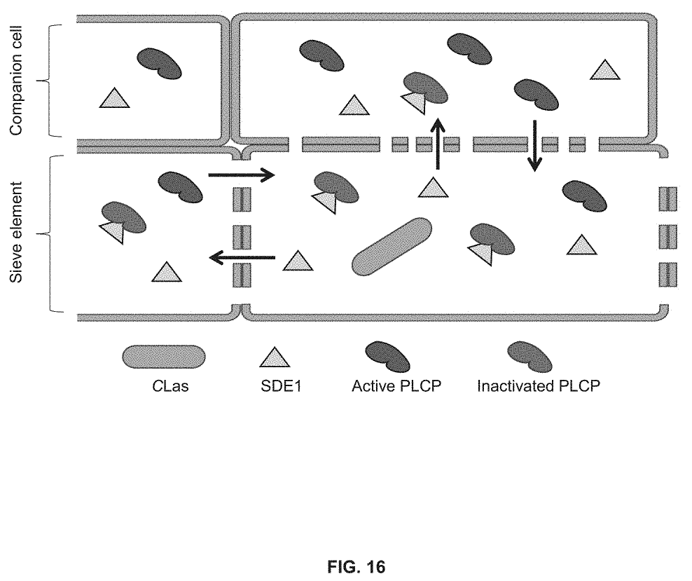

Bacterial, fungal, and oomycete pathogens as well as nematodes have all evolved effector proteins to suppress PLCP activities in order to promote infection^{20,21,25,32,37,38,39}. Cip 1 produced by the bacterial pathogen P. syringae is required for full virulence32. Similarly, the C. fulvum effector Avr2 and the Ustilago maydis effector Pit2 also play important roles during fungal infection of their respective plant hosts^{40,41}. SDE1 was able to partially substitute for Cip1 function during infection, indicating that it might similarly promote CLas infection in citrus. Although PLCPs are a major hub of effector targets, none of these effectors share sequence similarities, suggesting that they have evolved independently (through convergent evolution) to interfere with the activities of this important group of defense regulators. Phloem sieve tube elements are metabolically inactive and are supported by adjacent companion cells derived from the same mother cell⁴². PLCPs have been identified in phloem proteomic analyses of other plants, indicating that they could be directly secreted into sieve elements from adjacent companion cells^{43,44}. In CLas-infected citrus trees, increased accumulation of AALP, XBCP3, and SAG12 subfamily members was detected. It was found that SDE1 associates with multiple CsPLCPs in various subfamilies and there is a discordance between abundance and activity of three SAG12 members during CLas infection. SDE1 is potentially secreted into the phloem by CLas during infection, where it might act to suppress PLCP activity. SDE1 might also be able to move through the sieve elements and translocate into the companion cells via plasmodesmata to inhibit these important defense proteins (FIG. 16).

The findings described present a foundation for the development of HLB-resistant germplasm through genetic manipulation. Our findings showing that SDE1 does not inhibit RCR3 activity and thus fails to trigger Cf-2-dependent cell death in tomato illustrate the host specificity of these pathogen effectors and raise the possibility of engineering a similar immune receptor pathway to elicit defense responses upon effector-mediated inhibition of citrus PLCPs.

While not wanting to be limited by theory, it is thought that manipulating PLCP activity could be used to combat the inhibitory function of SDE1, which can lead to increased resistance of CLas. PLCP can be manipulated to result in increased activity, such as by either increasing expression or by manipulating the PLCPs to increase activity. PLCPs themselves could be excellent targets for genetic modification. In addition, it has been shown that overexpression of a specific PLCP gene in N. benthamiana increased disease resistance to P. infestans³⁹. Overexpression can be accomplished by expression cassettes or CRISPR-based promoter editing to manipulate PLCP gene expression. The end result can be a genetically-modified plant that could lead to urgently needed HLB resistance.

As noted above, plants that (1) express more than endogenous (native) levels of PLCP expression (which can be native or mutant PLCP proteins), (2) express mutant PLCP proteins that have reduced binding to SDE1 are provided. Reduced binding to SDE1 can be tested using an assay described in the EXAMPLES section.

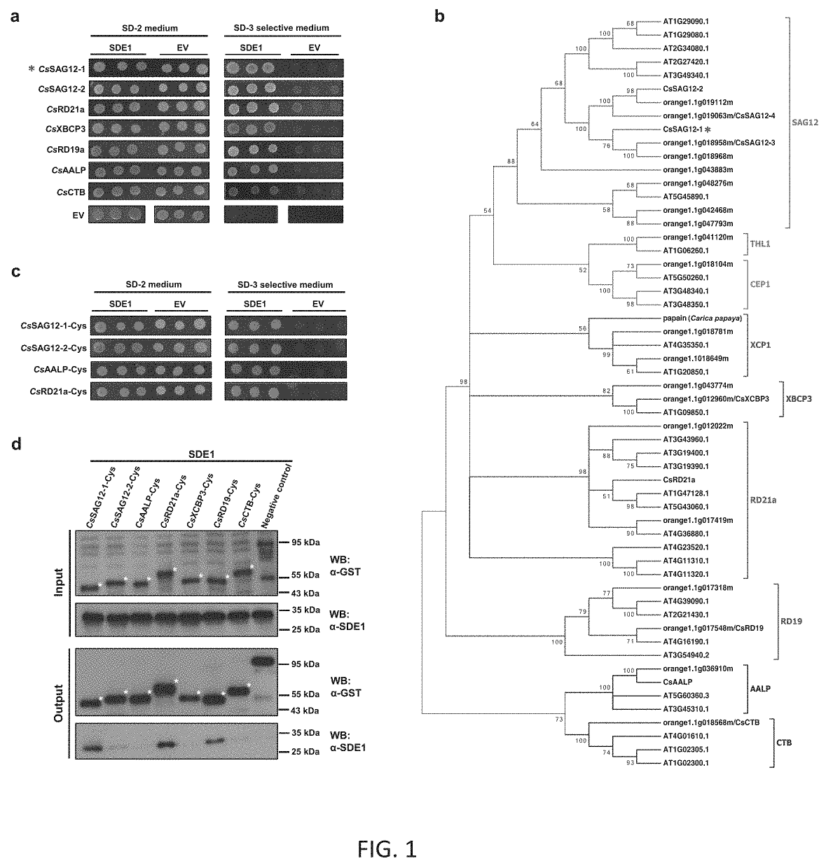

The present disclosure provides for heterologous expression of native or mutated PLCP polypeptides. In some embodiments, the citrus proteases so-manipulated comprise proteases selected containing an N-terminal signal peptide. In some embodiments, a PLCP polypeptide may be expressed that lacks a signal peptide native to the PLCP polypeptide. As used herein, the “mature region” of a PLCP polypeptide refers to a polypeptide lacking the N-terminal signal peptide sequence. In particular embodiments, the PLCP can comprise a protease selected from the following protease subfamilies: senescence-associated gene 12 (SAG12), thioredoxin h-like proteins (THL) (e.g., THL1), cysteine endopeptidases (CEP1), xylem cysteine proteasel (XCP1), xylem/bark cys peptidase 3 (XBCP3), Arabidopsis cysteine proteases RD21a, cysteine protease RD19, Arabidopsis aleurine-like proteases (AALP), and cathepsin-like proteases (CTB). In some embodiments, the protease comprises a protease from the SAG12 family, such as CsSAG12-1 (NCBI accession XM_006495158, previously GI #568885285), CsSAG12-2 (XM_006470229), CsSAG12-3 (XM orange1.1g018958), CsSAG12-4 (orange1.1g0119063) and CcSAG12-1 (Ciclev10005334). In some embodiments, the protease overexpressed comprise a protease from the XCBP3 family, such as CsXCBP3 (orange1.1g012960). In some embodiments, the protease overexpressed comprise a protease from the RD21a family, such as CsRD21a (XM_006473212). In some embodiments, the protease overexpressed comprise a protease from the RD19 family, such as CsRD19 (orange1.1g017548m). In some embodiments, the protease overexpressed comprise a protease from the AALP family, such as CsAALP (XM_006474664). In some embodiments, the protease overexpressed comprise a protease from the CTB family, such as CsCTB (orange1.1g018568). Other PLCP polypeptides include but are not limited to any of those provided in FIG. 1, Table 1 or Table 2, or as otherwise provided herein.

In some embodiments, the mutated or native PLCP polypeptide is substantially identical (e.g., at least 50%, 55%, 60%, 65%, 70%, 75%, 80%, 85%, 90%, 91%, 92%, 93′%, 94′% 95%, 96%, 97%, 98%, or 99%) to a wild-type PLCP polypeptide, e.g. SEQ ID NO:1 or SEQ ID NO:2; or comprises a mature region that is substantially identical to the mature region of a wild-type polypeptide. In some embodiments, the PLCP polypeptide is a mutated version of a native PLCP polypeptide comprising one or more (e.g., 1, 1-2, 1-3, 1-5, 1-10, etc.) amino acid substitutions compared to a wild type PLCP polypeptide. In some embodiments, the mutated PLCP polypeptide can be chosen such that the PLCP has reduced binding affinity (weaker or no binding) to SDE1. Binding activity can be performed, for example, using a yeast two-hybrid assay as described herein.

### Heterologous Expression

Once a polynucleotide encoding a native or mutated PLCP polypeptide is obtained, it can also be used to prepare an expression cassette for expressing the native or mutated PLCP polypeptide in a transgenic plant, directed by a promoter. In some embodiments, the expression cassette encodes a mature PLCP polypeptide joined to a heterologous signal peptide sequence. Increased expression of native or mutated PLCP polynucleotide is useful, for example, to produce plants that overexpress PLCP, thus enhancing resistance to HLB. In some embodiments, a native PLCP polypeptide is expressed from a promoter that generates more expression of the PLCP polypeptide that would occur in a native plant. This can involve, for example, linking a non-PLCP promoter to the coding sequence for the PLCP polypeptide. Alternatively, the endogenous PLCP promoter sequence can be altered (e.g., by one or more nucleotide change) to generate a higher expressing promoter. This latter method can involve introduction of a new expression cassette into the plant or can involve in vivo mutation, for example using a targeted nuclease (e.g., CRISPR, talens, zinc fingers) in combination with a second polynucleotide that is introduced by homologous recombination (and optionally a third targeting polynucleotide).

Any of a number of means can be used to drive native or mutated PLCP expression in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, the mutated or native PLCP polynucleotide can be expressed specifically in certain cell and/or tissue types within one or more organs (e.g., phloem, guard cells in leaves using a guard cell-specific promoter). Alternatively, the mutated or native PLCP polypeptide can be expressed constitutively. Some embodiments provide for a mutated PLCP nucleic acid operably linked to a promoter which, in some embodiments, is capable of driving the transcription of the PLCP coding sequence in plants. The promoter can be derived from plant or viral sources. The promoter can be constitutively active, inducible, or tissue specific. In the construction of recombinant expression cassettes, vectors, or transgenics as described herein, different promoters can be chosen and employed to differentially direct gene expression in some or all tissues of a plant or animal.

### Transgenic Plants

In any number of the embodiments described herein, transgenic plants are formed that comprise an introduced polynucleotide. In some aspects, plants comprising a mutated PLCP polypeptide comprising one or more amino acid substitutions as described herein are provided. In some embodiments, the plant is a transgenic plant comprising a recombinant expression cassette for expressing the native or mutated PLCP polypeptide in the plant. In some embodiments, a transgenic plant is generated that contains a complete or partial sequence of a polynucleotide that is derived from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

In some embodiments, the transgenic plant can have increased expression (e.g., at least 5%, 10%, 50% or more) of PLCP as compared to the wild-type plant. In some embodiments, the transgenic plant can have increased PLCP activity in the presence or absence or both of HLB. In some embodiments, the plants have increased HLB resistance or tolerance (reduced symptoms while still being infected).

### Method of Making Transgenic Plants Comprising Using Recombinant Expression Cassettes

In some embodiments, a recombinant expression vector comprising a native or mutated PLCP coding sequence driven by a promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the A. tumefaciens host will direct the insertion of the construct and adjacent marker into the plant cell DNA when the cell is infected by the bacteria. While transient expression of the constitutively active PLCP expressor is encompassed by the invention, generally expression of construction will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski⁶¹. Electroporation techniques are described in Fromm⁶². Ballistic transformation techniques are described in Klein⁶³. A. tumefaciens-mediated transformation techniques, including disarming and use of binary vectors, are well described in the scientific literature^{64,65}.

Transformed plant cells derived by any of the above transformation techniques can be cultured to regenerate a whole plant that possesses the transformed genotype and thus the desired phenotype such as enhanced resistance to HLB. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al.,⁶⁶ and Binding, Regeneration of Plants, Plant Protoplasts⁶⁷. Regeneration can also be obtained from plant callus, explants, organs, or parts thereof. Such regeneration techniques are described generally in Klee⁶⁸.

After the expression cassette is stably incorporated in transgenic plants and confirmed to be operable, it can be introduced into other plants by sexual crossing. Any of a number of standard breeding techniques can be used, depending upon the species to be crossed. The result can be a transgenic plant with increased expression or activity of PLCP as compared to the wild-type plant.

### Method of in Situ Alterations in Plants Via CRISPR/Cas9

Plant gene manipulations can be precisely tailored in non-transgenic organisms using the CR1SPR/Cas9 genome editing method. In this bacterial antiviral and transcriptional regulatory system, a complex of two small RNAs—the CRISPR-RNA (crRNA) and the trans-activating crRNA (tracrRNA)—directs the nuclease (Cas9) to a specific DNA sequence complementary to the crRNA⁶⁹. Binding of these RNAs to Cas9 involves specific sequences and secondary structures in the RNA. The two RNA components can be simplified into a single element, the single guide-RNA (sgRNA), which is transcribed from a cassette containing a target sequence defined by the user⁶⁹. This system has been used for genome editing in humans, zebrafish, Drosophila, mice, nematodes, bacteria, yeast, and plants⁷⁰. In this system the nuclease creates double stranded breaks at the target region programmed by the sgRNA. These can be repaired by non-homologous recombination, which often yields inactivating mutations. The breaks can also be repaired by homologous recombination, which enables the system to be used for gene targeted gene replacement^{71,72}. The PLCP expression mutations described in this application can be introduced into plants using the CAS9/CRISPR system. Accordingly, in some embodiments, instead of generating a transgenic plant, a native PLCP coding sequence in a plant or plant cell can be altered in situ to generate a plant or plant cell carrying a polynucleotide encoding a mutated PLCP polypeptide or comprising a mutated PLCP promoter as described herein.

The CRISPR/Cas system has been modified for use in prokaryotic and eukaryotic systems for genome editing and transcriptional regulation. The “CRISPR/Cas” system refers to a widespread class of bacterial systems for defense against foreign nucleic acid. CRISPR/Cas systems are found in a wide range of eubacterial and archaeal organisms. CRJSPR/Cas systems include type I, II, and III sub-types. Wild-type type H CRISPR/Cas systems utilize the RNA-mediated nuclease, Cas9 in complex with guide and activating RNA to recognize and cleave foreign nucleic acid. Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cvanobacteria, Firmicutes, Proteobacteria, Spirochaetes, and Thermotogae. An exemplary Cas9 protein is the Streptococcus pyogenes Cas9 protein. Additional non-limiting examples of Cas9 proteins and homologs thereof have been described in literature^{73,74,75,76,69}.

Accordingly, in one aspect, a method can be provided using CRISPR/Cas9 to introduce at least one of the mutation described herein into a plant cell. In some embodiments, a method of altering a (e.g., native) nucleic acid encoding the PLCP polypeptide in a plant is described. In some embodiments, the method can comprise introducing into the plant cell containing and expressing a DNA molecule having a target nucleic acid encoding PLCP polypeptide an engineered, non-naturally occurring Clustered Regularly Interspaced Short Palindromic Repeats (CRISPR)-CRISPR associated (Cas) (CRISPR-Cas) system. In some embodiments, the CRISPR-Cas system comprises one or more vectors comprising: (a) a first regulatory element operable in a plant cell operably linked to at least one nucleotide sequence encoding a CRISPR-Cas system guide RNA that hybridizes with the target sequence, and (b) a second regulatory element operable in a plant cell operably linked to a nucleotide sequence encoding a Type-II Cas9 protein, wherein components (a) and (b) are located on the same or different vectors of the system, whereby the guide RNA targets the target sequence and the Cas9 protein cleaves the DNA molecule, whereby at least one of the PLCP overexpression mutations described herein is introduced into the target nucleic acid encoding the PLCP polypeptide.

Other nuclease systems may also be used to introduce targeted modifications, e.g., a zinc-finger nuclease, a transcription activator-like effector nuclease (TALEN), or meganuclease-based system.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed considering the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice ant of the embodiments disclosed in the present disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. It should be understood that the disclosed subject matter is in no way limited to a particular methodology, protocol, and/or reagent, etc., as described herein. Various modifications or changes to or alternative configurations of the disclosed subject matter can be made in accordance with the teachings herein without departing from the spirit of the present specification.

### EXAMPLES

### Example 1.1

### Preparation of Plant Materials

Leaf and stem samples from symptomatic and asymptomatic trees were collected from mature Navel orange (Citrus sinensis) trees in a commercial orchard in Donna, Tex. and immediately frozen in liquid nitrogen. Samples were freeze-dried and sent on dry ice to the Contained Research Facility at the University of California, Davis for further processing. One-year-old Navels used for the quantitative PCR and protease inhibition assays were grown in a greenhouse at the University of California, Davis. The ambient temperature was kept at 23° C. with 72% relative humidity.

### Example 1.2

### Generation of SDE1-Transgenic Citrus

The 390 bp coding region of SDE1 without the signal peptide (1-24 aa) was amplified from DNA extracted from HLB-infected tissue using gene-specific primers with a start codon added to the 5′ end of the SDE1 forward primer. The PCR product was purified and cloned into pGEM-T Easy vector (Promega) and then sub-cloned into the binary vector erGFP-1380N. The recombinant vector was transformed into Agrobacterium tumefaciens strain EHA105 and then used for citrus transformation. Empty vector (EV) was used as a negative control. Agrobacterium-mediated transformation of etiolated grapefruit epicotyl segments⁴⁵ from the cultivar Duncan grapefruit was carried out. Epicotyls were soaked in Agrobacterium suspension for 1-2 minutes, cultured for two days, and then moved to a screening medium. Putative transformants were selected using kanamycin resistance and erGFP-specific fluorescence in putative transgenic lines was evaluated using a Zeiss SV11 epi-fluorescence stereomicroscope. Transgenic shoots were then micro-grafted in vitro onto one-month-old Carrizo citrange nucellar rootstock seedlings. After one month of growth in tissue culture, the grafted shoots were potted into a peat-based commercial potting medium and acclimated under greenhouse conditions.

### Example 1.3

### Yeast-Two-Hybrid Assays

A C. sinensis cDNA library was generated with total RNA extracted from healthy and CLas-infected tissues. The library was screened against SDE1 using a mating-based yeast-two-hybrid (Y2H) approach coupled with Illumina sequencing (performed by Qintarabio, Calif.). Sequences were analyzed by BLASTn using the NCBI database and top hits from C. sinensis were marked as potential SDE1-interacting proteins. Selected candidates from the Y2H screen were further tested using pairwise Y2H. The full-length cDNA of each potential SDElinteractor was cloned into the pGADT7 prey vector (Clontech) and transformed into yeast strain AH109 (Clontech) containing SDE1 on the bait plasmid pGBKT7. Transformation of the prey plasmids into AH109 containing pGBKT7 empty vector served as a negative control.

To test the interaction of SDE1 with PLCPs of various subfamilies, cDNA sequences of the PLCP representatives CsSAG12-1, CsSAG12-2, CsRD21a, CsRD19, CsAALP, CsXBCP3, and CsCTB, excluding their signal peptides, were cloned into pGADT7 and expressed in AH109. Signal peptides were predicted using SignalP 4.1 (organism group ‘Eukaryotes’; default D-cutoff values). For PLCP fragments encoding only the cysteine protease domain, full-length PLCP protein sequences were analyzed by SMART^{46,47} and sequences corresponding to the protease domains were cloned into pGADT7.

The experiments were repeated at least three times with similar results.

### Example 1.4

### Phylogenetic Analysis of PLCPs

Protein sequences of 31 PLCP genes from Arahidopsis thaliana22 and the annotated protein sequences from the entire sequenced genome of C. sinensis were downloaded from Phytozome (http address phytozome.jgi.doe.gov/pz/portal.html). Local BLASTp with an e-value of 1e-5 was used to search for PLCP homologs in C. sinensis using the AtPLCPs as query. To confirm that the resultant C. sinensis sequences are indeed homologous to the queried AtPLCPs, the BLASTp search was reversed. All PLCP protein sequences were aligned using MUSCLE v3.8.3⁴⁸. MEGA v6.06⁴⁹ was used to construct the maximum likelihood phylogenetic tree using the James-Taylor-Thorthon model and a bootstrap value of 100.

### Example 1.5

### In Vitro Pull-Down Assays

The protease domains of CsSAG12-1, CsSAG12-2, CsRD21a, CsRD19, CsAALP, CsXBCP3, and CsCTB were cloned into the pGEX-4T2 vector (GE Healthcare) and SDE1 was cloned into pRSF-Duet vector (gift from Dr. Jikui Song, University of California, Riverside). Vectors were transformed into E. coli BL21 cells (New England Biolabs) for protein expression. Total proteins were extracted from E. coli expressing the PLCPs and incubated with 25 μL glutathione resins (Thermo Scientific) for 1 hr at 4° C., followed by washing with TKET buffer (20 mM Tris-HCl, 200 mM KCl, 0.1 mM EDTA, 0.05% Triton X-100, pH 6.0). SDE1-expressing cell lysate was added to the PLCP-bound resins and incubated for 3 hrs at 4° C., followed by washing to remove non-specifically bound proteins. Washed resins were boiled in Laemmli sample buffer and the supernatants were used for gel electrophoresis and the subsequent immunoblotting. The enrichment of SDE1 proteins in PLCP-bound resins was detected using an anti-SDE1 antibodyl⁶ followed by goat anti-rabbit-HRP (Santa Cruz). Levels of PLCPs were determined using anti-GST (Santa Cruz) followed by goat anti-rabbit-HRP (Santa Cruz). After antibody incubation, the membranes were washed, and signals were developed using SuperSignal Chemiluminescent substrates (Thermo Scientific).

For the E-64 inhibition assay, glutathione resins with bound GST-tagged CsSAG12-1, CsRD21a, and RCR3³⁸ were incubated with either 200 μM E-64 as an inhibition treatment or TKET buffer as a control. Supernatant of SDE1-expressing cells was collected and incubated with the PLCP-bound resins for 3 hrs at 4° C. The resins were washed and enrichment of SDE1 detected by electrophoresis and subsequent immunoblotting as described above.

The experiments were repeated at least two times with similar results.

### Example 1.6

### In Vitro Protease Activity Assay with Papain

The EnzChek protease assay kit (Molecular Probes) was used to measure protease activity. Tag-free SDE1, E-64 (Sigma-Aldrich), and BSA (Gold Biotechnologies) at two different concentrations (100 and 500 nM) were mixed with papain (Sigma-Aldrich) at 100 μg/mL and added to 96-well Immulon plates (Thermo Scientific) containing BODIPY FL casein substrate. Papain with MES buffer alone served as a no treatment control for proteolytic activity and SDE2 (CLIBASIA_03230) at 300 nM served as an alternative CLas effector control. Reactions were allowed to perform for 1 hr at room temperature in the dark before fluorescence was measured using a Tecan Pro 2000 plate reader at 460/480 nm excitation/emission, with a gain value of 50. P-values were determined using a two-tailed student's t-test. SDE1 and SDE2 recombinant proteins were purified from E. coli using His60 Ni-NTA Superflow resins (Clontech). The purified SDE1 proteins were cleaved with Ubiquitin-like-specific protease 1 to remove the His-SUMO tag, generating tag-free SDE1 proteins.

The experiments were repeated at least three times with similar results.

### Example 1.7

### Activity-Based Protein Profiling (ABPP)

Papain (Sigma-Aldrich), Nicotiana bethamiana apoplastic fluids, and total citrus leaf extracts were pretreated with either buffer control, E-64, or SDE1 recombinant proteins. Total leaf extracts from SDE1-expressing transgenic citrus lines were pretreated with either 100 μM E-64 or buffer control. After pretreatment, the samples were incubated with a final concentration of 2 μM DCG-04²⁴ for 4 hrs at room temperature, followed by precipitation with 100% ice-cold acetone. Samples were centrifuged at 12,000×g, washed with 70% acetone, then centrifuged again. Precipitated products were re-suspended in 50 mM Tris buffer (pH 6.4) and either used directly for western blotting using Streptavidin-HRP conjugates (Thermo Scientific) or further enriched on streptavidinmagnetic beads (Thermo Scientific). For enrichment, samples were incubated with 25 μL streptavidin magnetic beads at room temperature for 1 hr, washed twice with 1% SDS, and eluted by heating for 5 min at 95° C. in Laemmli sample buffer with 13% β-mercaptoethanol50. The labeled proteins were separated using SDS-PAGE and active proteases were visualized by western blotting using Streptavidin-HRP conjugates (Thermo Scientific).

The experiments were repeated two times with similar results.

### Example 1.8

### Gene Expression Analyses Using qRT-PCR

One-year-old C. sinensis (Navel) trees grown in the greenhouse were sprayed with 2 mM salicylic acid (SA) or water with 0.02% of Silwet L-77 as an adjuvant. After 48 hours, fully expanded young leaves were harvested, flash frozen in liquid nitrogen, and stored at −80° C. A total of three trees (biological replicates) were analyzed for each treatment. Total RNA was extracted using a Trizol (Invitrogen)-based method. 1.5 mg RNA in a 20 μL reaction was used for cDNA synthesis using M-MLV reverse transcriptase (Promega). The CFX96 real-time PCR detection system (Bio-Rad) was used to assess PLCP gene expression. Quantitative reverse transcription PCR reactions used Bio-Rad SoFast EvaGreen Supermix according to the manufacturer's directions. Thermocyling began with a first step at 95° C. for 30 sec followed by 40 cycles alternating between 5 sec at 95° C. and 15 sec at 60° C. A melting curve was performed after the final cycle and ran 5 sec at 65° C. and 5 sec at 95° C. Gene expression was normalized to the Cyclooxygenase (COX, KF933043.1) gene⁵¹. All primers, gene names, and accession numbers are provided in Table 4.

This experiment was repeated three times with similar results.

### Example 1.9

### Citrus Imprint Assay

Freshly cut stems of CLas-infected (both symptomatic and asymptomatic) Rio Red grapefruit trees from a commercial orchard in Donna, Tex. and healthy (CLas-free) stems from grapefruit kept in a screen house were imprinted onto nitrocellulose membranes. CLas status was verified by qRT-PCR prior to imprinting. Imprinted membranes were then incubated with either anti-AALP (gift from Dr. Natasha Raikhel, University of California, Riverside) or anti-SDE1 antibodies¹⁶ and the corresponding proteins were detected using goat anti-rabbit-HRP secondary antibodies (Santa Cruz) and SuperSignal Chemiluminescent substrates (Thermo Scientific).

The experiments were repeated two times with similar results.

### Example 1.10

### Mass Spectrometry Analyses of PLCP Abundance and Activity

To assess for PLCP abundance, a total of 250 μg of uninfected and infected leaf extract was ground in 50 mM Tris (pH 6.8) and 2 μM DTT in a total reaction volume of 500 μL. Protein extracts were divided for the detection of activity (below) and PLCP abundance. Proteins were precipitated as described above for the ABPP assay. The protein pellet was re-suspended in 8 M urea in 100 mM ammonium bicarbonate (ABC). The samples were reduced and alkylated with 10 mM DTT and 30 mM of iodoacetamide (IAA) in 100 mM ABC for 1 hr, respectively. Samples were then diluted to a final concentration of 1 M urea by adding 100 mM ABC. Two micrograms of trypsin were added and the samples incubated overnight at 37° C. The tryptic digest was arrested by lowering the pH to ≤3 with formic acid. Peptide desalting and purification was performed with the MacroSpin C18 column protocol (The Nest Group).

To determine PLCP activity, the other half of the leaf extracts from above were incubated with a final concentration of 2 μM DCG-04 for 4 hrs at room temperature and precipitated as describes above for the ABPP assay, followed by further enrichment of the DCG-04 labeled products on streptavidin beads. Beads were washed three times with 50 mM ABC. Samples were reduced with 50 mM DTT for 1 hr at 60° C. and alkylated with 50 mM IAA for 1 hr at room temperature. Tryptic on-bead digests were performed with 250 ng of trypsin and the samples incubated at 37° C. overnight. The digests were arrested by adding 50 μL 60% acetonitrile/0.1% trifluoroacetic acid to the resins and incubating for 10 min at room temperature.

Peptides were submitted to the Proteomics Core of the Genome Center at the University of California, Davis for liquid chromatography-MS/MS. The LC-MS/MS system configuration consisted of a CTC Pal autosampler (LEAP Technologies) and Paradigm HPLC device (Michrom BioResources) coupled to a QExactive hybrid quadrupole Orbitrap mass spectrometer (Thermo Scientific) with a CaptiveSpray ionization source (Michrom BioResources). Peptides were analyzed by as described below⁵². Peptides were reconstituted in 2% acetonitrile and 0.1% formic acid and were washed on a Michrom C18 trap then were eluted and separated on a Michrom Magic C18AQ (200 μm×150 mm) capillary reverse-phase column at a flow rate of 3 μL/min. A 120 min gradient was applied with a 2% to 35% B (100% acetonitrile) for 100 min, a 35% B to 80% B for 7 min and 80% B for 2 min. Then a decrease of 80% to 5% B in 1 min followed by 98% A (0.1% formic acid) for 10 min. The QExactive was operated in Data-Dependent Acquisition (DDA) mode with a top 15 method. Spray voltage was set to 2.2 kV. The scan range was set to 350-1600 m/z, the maximum injection time was 30 ms and automatic gain control was set to 1×10⁶. Precursor resolution was set to 70,000. For MS/MS, the maximum injection time was 50 ms, the isolation window was 1.6 m/z, the scan range 200-2000 m/z, automatic gain control was set to 5×10⁴ and normalized collision energy was 27. The dynamic exclusion window was set to 15 sec and fragment product resolution was 17,500. An intensity threshold of 1×10⁴ was applied and the underfill ratio was 1%.

Peptide identification, analyses, and quantification: The raw data files were imported into MaxQuant v1.5.6.5⁵³ for label-free intensity based quantification. The database search engine Andromeda54 was used to search MS/MS spectra against the C. clementina and C. sinensis databases downloaded from Phytozome with a tolerance level of 20 ppm for the first search and 6 ppm for the main search. Trypsin/P was set as the enzyme and two missed cleavages were allowed. Protein N-terminal acetylation, Methionine oxidation, and NQ deamidation were set as variable modifications. The maximum number of modifications per peptide was set as five and contaminants were included. The ‘match between runs’ feature was checked with a match time window of 0.7 min and an alignment time window of 20 min. The FDR for protein level and peptide spectrum match (PSM) was set to 1%. The minimum peptide length was 6, minimum razor and unique peptides was changed to 0, and minimum unique peptides was set to 1. The minimum ratio count for protein quantification was set to 2.

To ensure that abundance and activity data were analyzed separately, the ‘Separate LFQ in parameter groups’ option in the global parameters tab was selected. This option allows MaxQuant to perform retention time alignments and calculate a normalization factor for abundance and activity separately. The other MaxQuant settings were left as default. The total peptide intensities for each replicate were summed and a normalization factor was calculated for each sample55. This normalization factor was applied based on the least overall proteome change. Peptide ratios between samples were then calculated to obtain a pair-wise protein ratio matrix between samples, which was subsequently used to rescale the cumulative intensity in each sample and provides the label-free intensity (LFQ) value⁵⁵. Raw MS data has been deposited in PRIDE (PXD008366)⁵⁶. The MaxQuant output file was imported into Perseus 1.5.015⁵⁷. Potential contaminants, reverse hits, and proteins identified only by modified peptides were excluded. The LFQ intensities were loge-transformed. Proteins not consistently identified in at least two out of the three replicates in at least one group were discarded. Missing values were substituted with values from a normal distribution of the obtained intensities using default settings (width 0.5, downshift 1.8). Differentially changing proteins were identified using a two-tailed Student's t-test. A p-value of less than 0.05 was used for truncation.

### Example 1.11

### Structural Model of CsSAG12-1

The protein sequence for the catalytic domain of CsSAG12-1 was submitted to ModWeb58 (http address compbio.ucsfedu/modweb/) using the default settings. The template used for CsSAG12-1 was CysEP from Ricinus communis (RCSB Protein Data Bank ID 1S4V) with 56% sequence identity. Molecular graphics images were produced using the UCSF Chimera package from the Resource for Biocomputing, Visualization, and Informatics at the University of California, San Francisco (supported by NIH P41 RR-01081). The Chimera interactive graphics modelling program was used to view and compare structures59.

### Example 1.12

### Pseudomonas syringae Infection Assay

The leaves of five-week-old Arabidopsis thaliana plants (ecotype Col-0) were infiltrated with bacterial suspensions at OD₆₀₀=0.0001 (approximately 1×10⁵ cfu/mL). The inoculated plants were transferred to a growth chamber (22° C., 16/8 hrs light/dark regime, 90% relative humidity), and the bacterial populations were determined as colony forming units (cfu) per cm2 three days after inoculation33. To induce SDE1 expression under the hopZ1a promoter, P. syringae strains were grown in M63 minimal medium containing 1% fructose60 at room temperature for 24 hrs. The bacterial cells were then collected by centrifugation and re-suspended in 10 mM MgSO₄ buffer for inoculation using needle-less syringes.

The experiments were repeated three times with similar results.

### Example 1.13

### Cf-2-Mediated Cell Death in Tomato

Full length Avr2 was synthesized using gBlocks Gene Fragments (Integrated DNA Technologies). Primers were designed to add a 6xHIS tag (SEQ ID NO: 3) at the N-terminus of the mature protein (no signal peptide) and the resultant fragment cloned into pFLAG-ATS (Sigma-Aldrich) (F: 5′-GTA AAG CTT CAC CAT CAC CAT CAC CAT GCC AAG AAA TTA-3′ (SEQ ID NO: 4), R: 5′-CTG AGA TCT CAA CCA CAA AGT CC-3′ (SEQ ID NO: 5)). The construct was transformed into E. coli BL21 for protein expression. Recombinant proteins were purified using Ni-NTA agarose (Qiagen) and dialyzed into water. SDE1 was purified as described above. Three different concentrations (10 nM, 100 nM, and 2 μM) of purified Avr2 and SDE1 recombinant proteins were syringe infiltrated into three-week-old leaves of tomato cultivar Moneymaker. The following near-isogenic lines were used25: Cf-2/RCR3^{pim}, Cf-2/rcr3-3, and Cf-0. Images were taken 7 days after infiltration.

The experiments were repeated two times with similar results.

### Example 1.14

### Statistical Data Analysis

The biological data reported was analyzed as follows using SAS JMP Pro v13.0. To test for normal distribution of the collected data, normal quantile plots were inspected and Shapiro-Wilk goodness-of-fit tests were performed. To ensure that the variances are equal, the Levene's test was used. When comparing a test group to a control group, a two-sided Student's t-test was used. The significance values are reported as follows: *=p<0.05, **=p<0.01, and ***=p<0.001. When comparing the means of multiple groups, a one-way ANOVA followed by Tukey's HSD post hoc test was performed. Significant differences between groups (p<0.05) are denoted with different letters.

### Example 1.15

### Antibodies and Chemicals

Streptavidin-HRP used for ABPP of PLCPs was purchased from ThermoFisher (Cat No. 21130) and used in 1:1000 dilution. Antibodies used in this study include Goat-anti-Rabbit IgG-HRP (Santa Cruz, Cat No. SC2004, used in 1:5000 dilution), Anti-AALP (anti-serum gifted from Dr. Natasha Raikel in Ref 30, used in 1:1000 dilution), Anti-SDE1 (polyclonal antibody generated in Ref 16, used in 1:1500 dilution), Anti-GST (Santa Cruz, Cat No. SC138, used in 1:2000 dilution). Anti-HA high affinity (Roche, Cat No. 11867423001, used in 1:1:500 dilution). Goat-anti-Rat IgG-HRP (Santa Cruz, Cat No. SC2065, used in 1:5000 dilution).

### Example 2

### Showing SDE1 Associates with Citrus Papain-Like Cysteine Proteases

SDE1 is unique to CLas with no homologs in other organisms¹⁶. It is found in all sequenced CLas isolates from various geographic regions and its expression was detected from CLas-infected citrus varieties including limes, sweet oranges, and grapefruits^{15,16}. To understand the potential virulence function of SDE1 in citrus, we performed sequencing-based yeast-two-hybrid (Y2H) screening using a Citrus sinensis cDNA library to identify candidate SDE1-interacting proteins (Table 1). A selection of these candidates was further examined using a pair-wise Y2H assay. Of the six evaluated candidates, the C. sinensis protein annotated as ‘xylem cysteine protease 1’ (NCBI accession XM_006495158, previously GI #568885285) was confirmed by Y2H as interacting with SDE1 (FIG. 1, panel a).

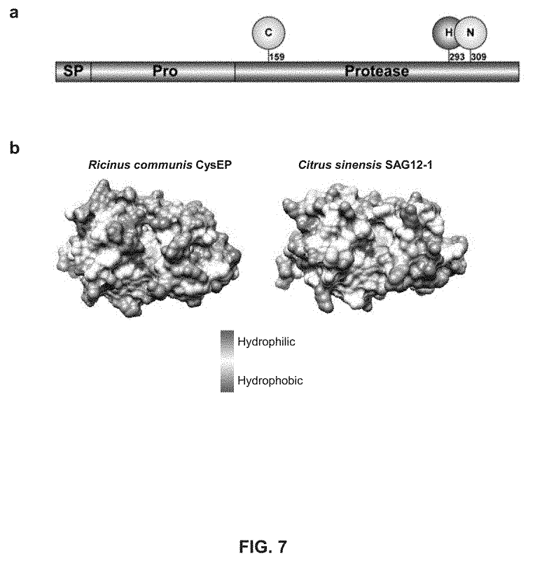

Xylem cysteine protease 1 is a member of the papain-like cysteine protease (PLCP) family. PLCPs share a conserved protease domain including a catalytic triad consisting of cysteine, histidine, and asparagine¹⁹ (FIG. 7, panel a). The canonical PLCPs have a pro-domain that must be autocatalytically processed for activity. The pre-proteases often contain an N-terminal signal peptide to ensure their entrance into the endomembrane system and subsequent function in the apoplast, vacuole, or lysosomes (FIG. 7, panel a). Previous reports have shown that PLCPs contribute to plant defense during bacterial, oomycete, and fungal infection^{19,20,21}. Search of the C. sinensis genome revealed 21 canonical PLCPs that can be classified into nine subfamilies based on their homology to the previously categorized Arabidopsis PLCPs²² (FIG. 1, panel b). Based on our phylogenic analysis, XM_006495158 belongs to the SAG12 subfamily and is hereafter referred to as CsSAG12-1. Structural modeling using CysEP, a castor oil (Ricinus communis) PLCP involved in programmed cell death (PCD)²³, indicates that CsSAG12-1 adopts a similar fold in the protease domain, further supporting it as a PLCP (FIG. 7, panel b).

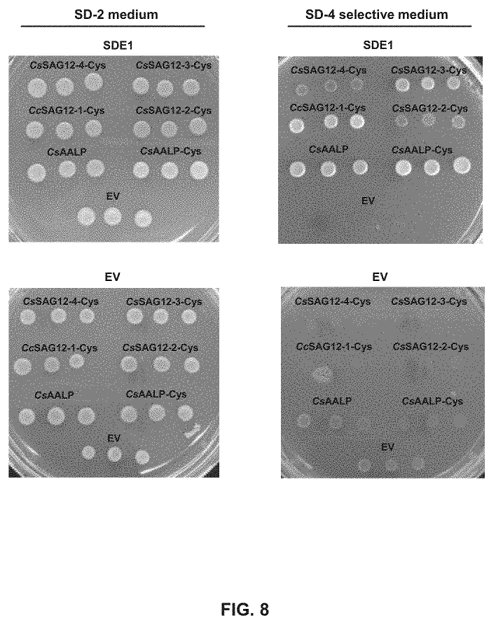

Since PLCPs share a conserved catalytic domain, we examined whether SDE1 could also associate with PLCPs from other subfamilies. Representatives from five additional PLCP subfamilies, CsXCBP3 (orange1.1g012960), CsRD21a (XM_006473212), CsRD19 (orange1.1g017548), CsAALP (XM_006474664), and CsCTB (orange1.1g018568), were tested. Remarkably, all of them were able to interact with SDE1 in yeast (FIG. 1, panel a). Furthermore, a second member of the SAG12 subfamily, CsSAG12-2 (XM_006470229), also interacted with SDE1 (FIG. 1, panel a). The observation that SDE1 interacts with members from multiple PLCP subfamilies suggests that it may associate with the conserved protease dom″ain. Indeed, the protease domains of CsSAG12-1, CsSAG12-2, CsRD21a, and CsAALP are sufficient to mediate interaction with SDE1 in yeast (FIG. 1, panel c). In addition, SDE1 interacted with the protease domains of three other members from the SAG12 subfamily, i.e. CsSAG12-3 (orange1.1g018958), CsSAG12-4 (orange1.1g019063), and CcSAG12-1 (Ciclev10005334, a PLCP from C. clementina) in yeast (FIG. 8).

In order to determine if SDE1 can directly interact with citrus PLCPs, we conducted in vitro pull-down assays using recombinant proteins expressed and purified from Escherichia coli. The protease domains of the PLCPs were tagged with GST at the N-terminus and the recombinant proteins were incubated with HIS-tagged SDE1 in excess. The protein complexes were immunoprecipitated using glutathione beads and enrichment of SDE1 was detected by western blotting. Our results show that SDE1 co-precipitated with the protease domains of CsSAG12-1, CsSAG12-2, CsRD19, and CsRD21a (FIG. 1, panel d). Although CsAALP, CsXCBP3, and CsCTB were able to interact with SDE1 in yeast, these interactions were not detected in the in vitro pull-down assay. This could be, at least in part, due to the poor solubility of the recombinant GST-PLCP proteins when produced in E. coli. The cysteine residues within the protease domains have the potential to form disulfide bonds^{19,22}, which may have resulted in incorrect folding and/or low solubility of these normally secreted PLCPs when expressed in the cytoplasm. Another possibility is that the pull-down assay is more stringent (and thus, less sensitive) in monitoring particular SDE1-PLCP interactions than Y2H. Nonetheless, these experiments strongly suggest that SDE1 can interact with multiple PLCPs belonging to different subfamilies through the conserved cysteine protease domain.

TABLE 1
Top Candidate from Y2H screening.

| | NCBI | Y2H |
|---|---|---|
| SDE1-interacting candidate | Sequence ID | Positive |
| Diacylyglycerol (DAG) protein, chloroplastic | XM_006475194 | No |
| E3 ubiquitin-protein ligase RNF12-B | XM_006489385 | No |
| Putative E3 ubiquitin-protein ligase XBAT31 | XM_006488247 | No |
| Heavy-metal-associated domain-containing | XM_006467144 | No |
| protein | | |
| Calcyclin-binding protein | XM_006467400 | No |
| Xylem cysteine proteinase 1 | XM_006493578 | Yes |
| Aspartic Proteinase | | N/A |
| Vignain-Thiol protease | | N/A |
| Catalase isozyme | | N/A |

### Example 3

### Showing SDE1 Inhibits PLCP Activity

Knowing SDE1 interacts with PLCPs through the protease domain, we next examined whether it could inhibit their proteolytic activity. Several assays were used to measure the proteolytic activities of PLCPs in the presence of SDE1. In all these assays, the chemical inhibitor E-64, which forms a covalent bond with the catalytic cysteine of the PLCP protease domain, was used as a positive control24.

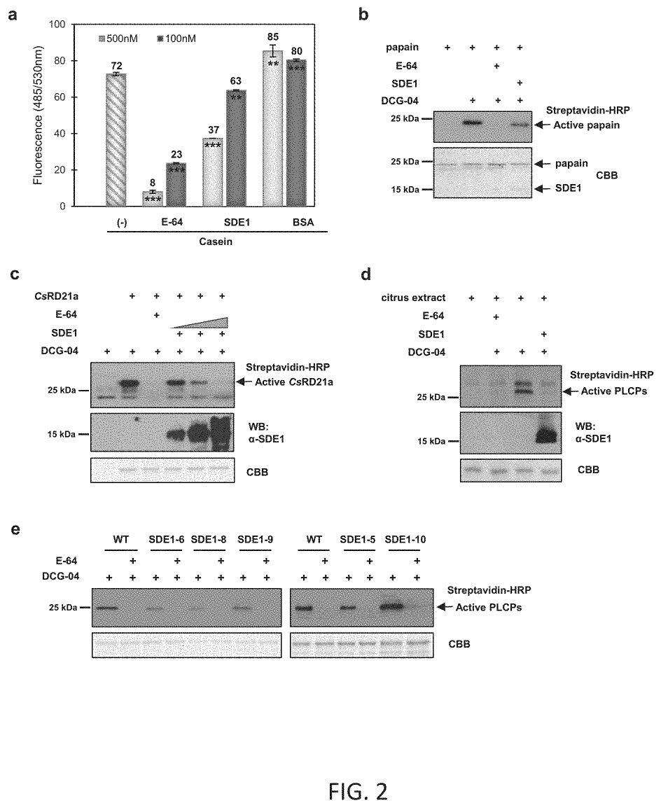

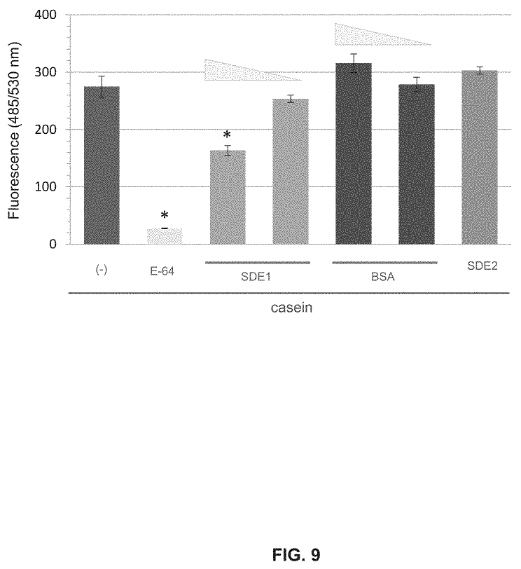

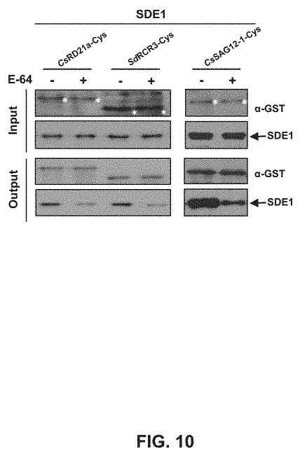

First, the inhibitory effect of SDE1 on the proteolytic activity of papain, a PLPC from papaya²² was examined. Fluorescein-labeled casein was used as a substrate which, upon cleavage by papain, releases a fluorescent signal that can be quantified using a fluorometer. Our results show that SDE1 inhibited substrate cleavage by papain in a dose-dependent manner (FIG. 2, panel a). Using 100 and 500 nM purified SDE1 protein, the proteolytic activity of papain was decreased by 12% and 49%, respectively, when compared to papain alone. This inhibitory effect is significant, although weaker compared to that of E-64, which reduced protease activity at the same concentrations by about 68% and 85%. As a negative control, addition of BSA or another CLas effector, termed SDE2, which does not interact with PLCPs, did not reduce the protease activity of papain (FIG. 2 panel a; FIG. 9). Next, we examined whether SDE1 binds near the catalytic site of PLCPs, if so, its interaction with PLCPs should be blocked by pre-incubation with E-64. We conducted in vitro pull-down assays with or without E-64 using the protease domains of two citrus PLCPs, CsSAG12-1 and CsRD21a, that can be pulled down by SDE1 in the absence of E-64 (FIG. 1, panel d). We also included a third PLCP, Resistance to Cladosporium falvum 3 (RCR3), which is a member of the tomato SAG12 subfamily and is known to be inhibited by the Avr2 effector from the fungal pathogen C. fulvum25. The protease domains of these PLCPs were expressed in E. coli and enriched using GST affinity resins. PLCP-bound resins were pre-incubated with 200 μM E-64 and the enrichment of SDE1 with the resins was examined by western blotting. Co-precipitation of SDE1 with all three PLCPs was reduced in the presence of E-64, suggesting that SDE1 binds near the catalytic cysteine bound by E-64, resulting in a steric hindrance around the active site (FIG. 10). Since SDE1-PLCP interactions were not completely abolished by the addition of E-64, it is likely that SDE1 does not directly bind to the catalytic cysteine residue. Rather, SDE1 might block the catalytic cleft to prevent access to substrates, thus inhibiting proteolytic activity. Alternatively, the binding of E-64 to the catalytic cysteine could result in conformational changes of the protease, and therefore, partially interfere with SDE1's interaction with the PLCPs.

Finally, the protease activity of SDE1-interacting PLCPs was directly measured using activity-based protein profiling (ABPP) where DCG-04, a biotinylated derivative of E-64, is used as a probe²⁴. Since E-64 only binds to the active form of cysteine proteases, western blots using streptavidin conjugated with horseradish peroxidase (HRP) can detect DCG-04-labled PLCPs via biotin, and the signal intensity reflects their activity level. First, we examined ABPP of papain in the presence of SDE1 or E-64. Our results showed that pre-incubation with SDE1 at 1.6 μM was able to reduce DGC-04 labeling by about 53%, demonstrating that SDE1 suppresses the protease activity of papain in vitro (FIG. 2, panel b). Pre-incubation of papain with E-64 (10 μM) completely abolished the DCG-04 labeling, which is consistent with the results from the in vitro protease activity assay using the fluorescein-labeled substrate.

ABPP was also conducted in a semi-in vitro assay using recombinant SDE1 protein purified from E. coli and PLCPs expressed in plant tissues. To this end, full-length CsRD21a was transiently expressed in Nicotiana benthamiana leaves. Using the native N-terminal secretion signal, CsRD21a was secreted into the apoplast as shown by coomassie brilliant blue (CBB) stain comparing control apoplastic fluids from wild-type N. benthamiana to those transiently expressing CsRD21a (FIG. 2, panel c). CsRD21a could be labeled by DCG-04, suggesting that it is an active enzyme. A reduction of CsRD21a activity was observed with the addition of SDE1 in a dose-dependent manner using 0.8, 1.6, or 3.2 μM purified proteins (FIG. 2, panel c). We then determined whether SDE1 could inhibit other PLCPs in citrus. Total proteins were extracted from leaves of Navel oranges (C. sinensis). We induced PLCP accumulation by spraying the leaves with 2 mM of the defense hormone salicylic acid (SA)²⁶, followed by total protein extraction and incubation with purified SDE1 protein. In this experiment, we further purified and concentrated the labeled PLCPs using streptavidin beads. Immunoblots using streptavidin-HRP showed that PLCP activity was greatly decreased after incubation with 120 nM SDE1, and completely inhibited with 25 μM E-64 (FIG. 2, panel d). Together, these results demonstrate that SDE1 suppresses the protease activity of CsRD21a and possibly other citrus PLCPs natively in the plant cells.

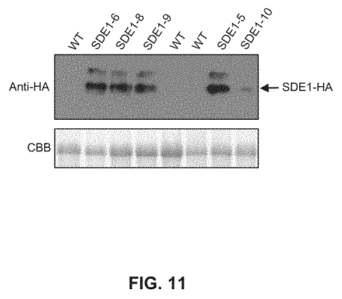

To further demonstrate that PLCPs are the in vivo targets of SDE1 in citrus, we generated transgenic seedlings of Duncan grapefruit expressing SDE1 (without the N-terminal 1-24 aa that corresponds to a secretion signal peptide) under the cauliflower mosaic virus 35S promoter. Total protein extracts from leaf tissues of one-year-old seedlings were labeled with DCG-04 and the levels of active PLCPs were examined by western blotting using streptavidin-HRP. Our results show reduced PLCP activities in four independent SDE1-expressing lines (SDE1-5, SDE1-6, SDE1-8, and SDE1-9), relative to an untransformed control (FIG. 2, panel e). We confirmed that these lines were indeed producing SDE1 proteins using western blotting (FIG. 11). In addition, the transgenic line SDE1-10 exhibited little to no SDE1 protein accumulation (FIG. 11) which correlated with a lack of reduction in protease activity in this line (FIG. 2., panel e). Taken together, these data strongly suggest that SDE1 can inhibit the protease activity of PLCPs in citrus.

### Example 4

### Showing Citrus PLCPs Accumulate During SA Treatment and Infection

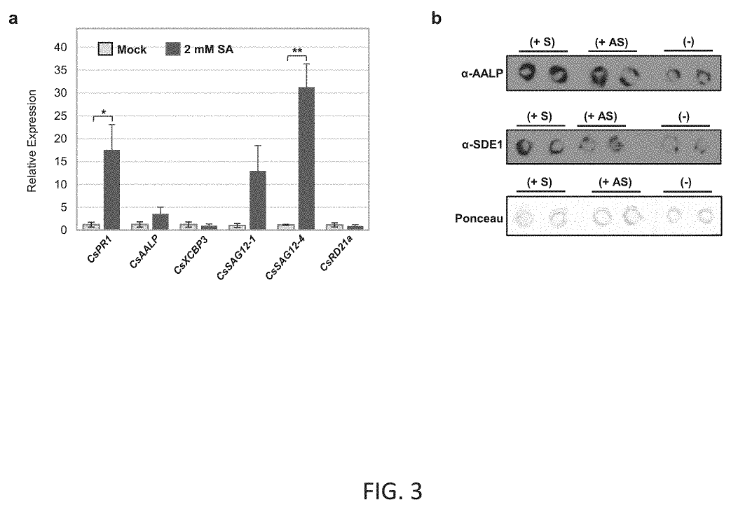

In order to determine if PLCPs are involved in defense-related responses in citrus, we looked at PLCP expression changes in both defense-induced and CLas-infected citrus. To activate defense signaling, leaves of Valencia oranges (C. sinensis) were sprayed with 2 mM salicylic acid (SA)²⁶. The transcript abundance of five CsPLCP genes was then determined by quantitative RT-PCR (qRT-PCR). Upon SA treatment, we detected an increase in the expression of Pathogenesis-related gene 1 (CsPR1)²⁷, which is a commonly used marker for the SA response. Although the magnitude of induction varied across trees, we consistently found a PLCP gene belonging to the SAG12 subfamily (CsSAG12-4) to be significantly up-regulated upon SA treatment (FIG. 3, panel a). CsSAG12-1 and CsAALP also showed a trend of increased expression in response to SA treatment, although the induction was not statistically significant. In addition, citrus PLCP genes have been shown to be transcriptionally induced in response to CLas infection^{28,29}. Analysis of publicly available transcriptome data^{28,29} found genes encoding CsPLCPs of several subfamilies including, but not limited to, SAG12, RD21a, and AALP to be up-regulated during CLas-infection (Table 2). These results indicate that citrus PLCPs may act as defense proteases in CLas-infected trees.

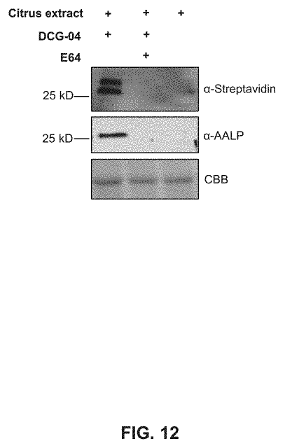

Since CLas is a phloem-colonizing bacterium, we next assessed whether SDE1 and PLCPs could both be detected in the phloem sap of infected citrus trees. For this purpose, we performed direct tissue imprints using anti-SDE1¹⁶ or anti-AALP30 antibodies, respectively. We monitored AALP as a representative of PLCPs in this experiment due to the availability of the antibody, although induction of CsAALP by SA treatment was not as robust as induction of the SAG12 subfamily members (FIG. 3, panel a). The specificity of the anti-AALP antibody was verified using DCG-04 labeling followed by western blotting (FIG. 12). Young stems from CLas-infected and healthy (i.e. CLas-free) trees of Rio Red grapefruit (Citrus paradisi) were freshly cut and imprinted onto nitrocellulose membranes, which were then incubated with either anti-SDE1 or anti-AALP. For the CLas-infected trees, we examined both symptomatic and asymptomatic tissues, which presumably represent late and early infection stages, respectively, as suggested by the bacterial titers. Our results show that while SDE1 was only present in the infected tissues, AALPs were detected from both healthy and infected tissues (FIG. 3, panel b). However, the signals representing AALPs were stronger in the infected stems, both symptomatic and asymptomatic, compared to those from the healthy stems. This is consistent with the increased abundance of PLCP genes revealed by qRT-PCR of SA-treated citrus (FIG. 3, panel a) and the analysis of previous transcriptome data (Table 2). Furthermore, similar to SDE1, the AALP signals were mainly detected from the bark layers, which is enriched with phloem cells.

TABLE 2
Analysis of published transcriptome data showing differentially expressed PLCPs in
CLas-infected C. sinensis.

| | PLCP | | | |
|---|---|---|---|---|
| Gene ID in C. sinensis | subfamily | Log₂FC¹ | FDR² | Source |
| orange1.1g018568m | CTB | −1.93764 | N/A | Martinelli et. al. (2012) |
| | | | | RNA-seq |
| orange1.1g047264m | XCP1 | −1.07107 | N/A | Martinelli et. al. (2012) |
| | | | | RNA-seq |
| orange1.1g017419m | RD21a | 1.1742 | N/A | Martinelli et. al. (2012) |
| | | | | RNA-seq |
| orange1.1g012960m | XBCP3 | 1.43348 | N/A | Martinelli et. al. (2012) |
| | | | | RNA-seq |
| orange1.1g036910m | AALP | 1.54139 | N/A | Martinelli et. al. (2012) |
| | | | | RNA-seq |
| orange1.1g024783m | RD21a | 1.59572 | N/A | Martinelli et. al. (2012) |
| | | | | RNA-seq |
| orange1.1g019063m | SAG12 | 1.97224 | N/A | Martinelli et. al. (2012) |
| | | | | RNA-seq |
| orange1.1g019112m | SAG12 | 2.6825 | N/A | Martinelli et. al. (2012) |
| | | | | RNA-seq |
| orange1.1g018781m | XCP1 | 0.822224 | N/A | Martinelli et. al. (2012) |
| | | | | RNA-seq |
| orange1.1g017318m | RD19 | 0.916496 | N/A | Martinelli et. al. (2012) |
| | | | | RNA-seq |
| orange1.1g012960m | XCBP3 | 0.812 | 3.08E−15 | Kim, J.-S. et. al. (2009) |
| | | | | Microarray |
| orange1.1g017419m | RD21a | 0.815 | 0.0438 | Kim, J.-S. et. al. (2009) |
| | | | | Microarray |
| orange1.1g018104m | CEP1 | 1.384 | 1.82E−06 | Kim, J.-S. et. al. (2009) |
| | | | | Microarray |
| orange1.1g018958m | SAG12 | 3.192 | 3.08E−15 | Kim, J.-S. et. al. (2009) |
| orange1.1g018968m | | | | Microarray |

¹Log₂ fold-change
²False discovery rate
Martinelli, F. et al. Transcriptome Profiling of Citrus Fruit Response to Huanglongbing Disease. PLOS ONE 7, e38039, 2012
Kim, J.-S., Sagaram, U. S., Burns, J. K., Li, J.-L. & Wang, N. Response of sweet orange (Citrus sinensis) to ‘Candidatus Liberibacter asiaticus’ infection: microscopy and microarray analyses. Phytopathology 99, 50-57 (2009)

### Example 5

### Uncoupling PLCP Abundance and Activity During CLas Infection

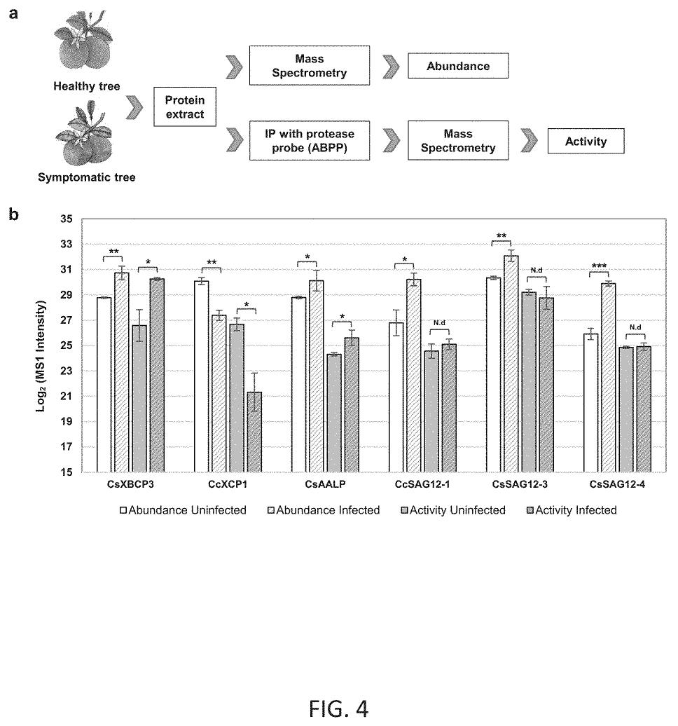

During pathogen recognition, PLCP abundance is usually increased alongside their activity³¹. Previous studies have demonstrated that various pathogens can selectively inhibit PLCPs in their specific plant hosts to facilitate disease progression¹⁹. To determine if this occurs during CLas infection, we performed comparative proteomics using tissues from mature Navel orange (C. sinensis) trees grown in a Texas grove. Leaves from CLas-infected (symptomatic) trees were collected. As a control, uninfected leaf samples were collected from trees held in a screenhouse that was consistently tested for CLas by qRT-PCR. PLCP abundance in total protein extracts was determined by mass spectrometry (MS), while active protease levels were also analyzed in the same samples using ABPP coupled with MS quantification (FIG. 4, panel a). We were able to detect multiple PLCP subfamilies by MS (FIG. 4, panel b). Among them, members of the AALP and XBCP3 subfamilies significantly increased in abundance as well as activity in infected trees compared to uninfected controls. A member of the XCP1 subfamily exhibited decreased abundance as well as activity in infected trees. Interestingly, the abundance and activity did not correlate for the three SAG12 subfamily members in this analysis. The abundance of CcSAG12-1, CsSAG12-3, and CsSAG12-4 significantly increased in infected trees, whereas their activity remained unchanged (FIG. 0066 4, panel b). This result indicates that these SAG12 subfamily members are potentially involved in citrus defense responses and that their activities might be inhibited by CLas. While it is tempting to speculate that SDE1 contributes solely to the inhibition of these PLCPs, the observed effect could be due to the concerted action of several effector proteins and/or other virulence factors of CLas, in addition to SDE1.

### Example 6

### Showing SDE1 Promotes Bacterial Infection

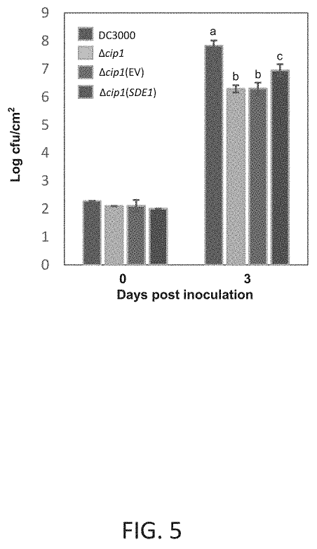

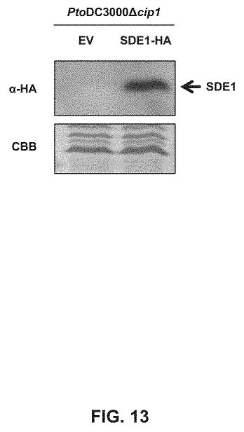

Despite substantial research efforts, CLas has not been successfully cultivated. In order to explore the potential contribution of SDE1 to bacterial virulence, we employed another gram-negative bacterial pathogen, Pseudomonas syringae, as a surrogate. In particular, P. syringae pv. tomato strain DC3000 (PtoDC3000) was previously reported to produce a Sec-secreted protein called Cipl, which can inhibit the protease activity of tomato C14, a member of the RD21a subfamily of PLCPs³². A cip1 knockout mutant of PtoDC3000 exhibited reduced virulence, indicating that Cipl contributes to bacterial infection, likely through its inhibitory effect on PLCP activities³². We examined whether SDE1 could complement the Cip1 virulence activity that was lost in the knockout mutant of PtoDC3000. SDE1 (full-length, containing its native Sec-secretion signal) was expressed in PtoDC3000Δcip1 under the promoter of hopZ1a, a type III-secreted effector that is activated during infection³³. We were able to detect SDE1 protein in the supernatant of induced bacterial cell cultures, confirming that it was secreted by P. syringae (FIG. 13). PtoDC3000, PtoDC3000Δcip1, and two PtoDC3000Δcip1 strains either expressing SDE1 or transformed with the empty vector (EV) were used to inoculate mature leaves of Arabidopsis thaliana ecotype Col-0 and the bacterial populations were determined three days post inoculation. The results show that while PtoDC3000Δcip1 and the EV control exhibited strong reductions in virulence compared to wild-type PtoDC3000, expression of SDE1 partially, but significantly, complemented this virulence deficiency (FIG. 5). Collectively, these results suggest that SDE1 promotes bacterial infection, likely by inhibiting PLCP activity in plant hosts.

### Example 7

### Showing SDE1 Does Not Inhibit RCR3 Activity in Solanaceous Plants

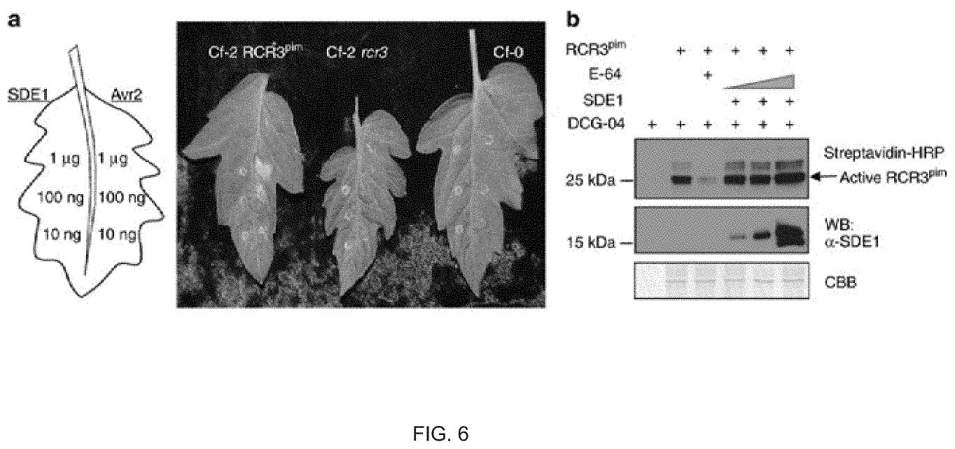

In tomato, inhibition of RCR3 activity by the C. fulvum effector Avr2 activates Cf-2-mediated immune responses, including programmed cell death, conferring resistance to the fungal pathogen²⁵. SDE1 interacts with RCR3 in vitro (FIG. 10). We therefore tested whether SDE1 can likewise trigger Cf-2-mediated cell death in tomato. To this end, we infiltrated near-isogenic lines of tomato cultivar Moneymaker²⁵ containing either Cf-2 and RCR3 (Cf-2 RCR3^{pim}), Cf-2 only (Cf-2 rcr3-3), or lacking Cf-2 (Cf-0) with purified SDE1 protein (FIG. 6, panel a). As a control, we infiltrated the same leaves with purified Avr2 protein. As expected, Avr2 triggered cell death in a Cf-2- and RCR3-dependent manner 7 days post infiltration; in contrast, no cell death was observed from SDE1-infiltrated areas even at high protein concentrations (FIG. 6, panel a).

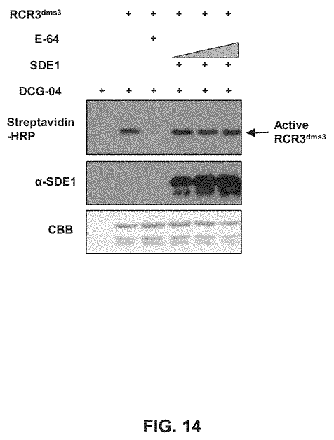

Next, we tested if this lack of cell death was due to SDE1 not being able to inhibit RCR3. We performed ABPP using RCR3 from tomato (RCR3^{pim}) and from the wild potato species Solanum demissum (RCR3^{dms3})³⁸. Unlike with CsRD21a, SDE1 was unable to inhibit the activity of either of the RCR3 proteins (FIG. 2, panel c; FIG. 6, panel b; FIG. 14). This result indicates that the lack of Cf-2-mediated cell death in response to SDE1 is likely due to the inability of the CLas effector to inhibit the protease activity of RCR3 from these non-host plants and illustrates the host-specific function of SDE1.

### Example 8

### SDE1 Promotes Bacterial Infection in Transgenic Citrus Plants

Transgenic citrus (grapefruit) plants expressing the CLas effector SDE1 were generated and inoculated with CLas. The CLas bacterial titer was monitored each month by quantitative PCR (presented as Ct values, which are inversely proportional to the amount to target nucleic acid). Table 3 below shows results obtained about 5 months after infection. It is normal to see CLas establishment in citrus between 6-12 months after inoculation. Ct values were measured as per 100 ng total DNA. “Not detectable” means Ct values are too high to be quantified (reflecting no CLas DNA was detected). A Ct value difference of 4 (34.56-30.53) reflects ˜20-50 fold difference in bacterial titer. In this experiment, there were large variations between different transgenic SDE1-expressing citrus plants, which could be due to different expression levels of SDE1 in these independent transgenic lines. It is also normal to see variations in CLas infection assays. The results in Table 3 demonstrate a significantly higher CLas bacterial titer (i.e., lower Ct values) in SDE1-expressing citrus plants, which indicates that SDE1 promoted HLB development and that SDE1 is an important virulence factor that makes citrus susceptible to the bacterial pathogen. The data thus support that blocking SDE function, which is to inhibit the protease activity of PLCPs, enhances citrus resistance.

TABLE 3
CLas bacterial titer, Ct values

| Line | SDE1-expressing citrus | Line | Control |
|---|---|---|---|
| 1 | 34.36 ± 0.16 | 1 | 34.66 ± 1.27 |
| 2 | 21.80 ± 0.39 | 2 | Not detectable |
| 3 | 36.26 ± 0.59 | 3 | 34.45 ± 1.21 |
| 4 | 29.69 ± 0.56 | 4 | Not detectable |
| Mean | 30.53 | | 34.56 |

### Example 9

### Expression of PLCP Transcripts in Citrus

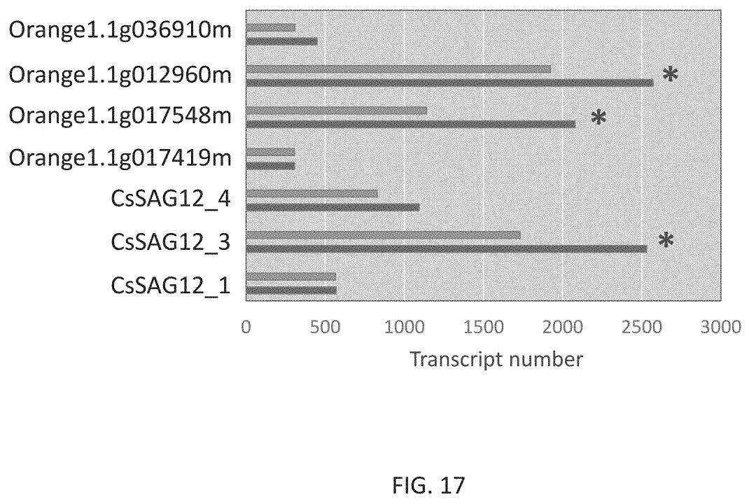

Transcript levels of 21 PLCP genes were examined in seven citrus varieties, including HLB-tolerant varieties (Sugar Belle, Australian finger lime, and Carrizo) and susceptible varieties (Clementine, Sweet oranges, Pumelo and Alemow) by Nanostring. FIG. 17 shows a comparison of the average transcript levels of several representative PLCP genes in tolerant varieties (lower bar for each variety) vs susceptible varieties (upper bar for variety). Significantly higher expression of three genes was observed in the tolerant varieties: CsSAG12_3 (orange1.1g018958m), orange1.1g017548m, and orange1.1g012960m.

Transcript expression analysis (FIG. 17) showed that CsSAG12-3 (orange1.1g018958m) exhibited a generally higher expression level in HLB-tolerant citrus varieties than susceptible varieties. This evidence supports a correlation between higher expression levels of CsSAG12-3 and tolerance to HLB. An additional PLCP, CsRD19 (orange1.1g017548m), also has a generally higher expression level in HLB tolerant varieties than susceptible varieties. CsRD19 was not detected in the Mass Spectrometry analysis described herein; however, it was shown that SDE1 interacts with CsRD19 (FIG. 1).

Transgenic citrus plants over-expressing (under CaMV 35S promoter) CsSAG12-1 (XM_006495158) or CsSAG12-3 (orange1.1g018958m) have been generated. These two genes were shown to be induced by pathogen infection and inhibited by SDE1. As noted above, CsSAG12-3 (orange1.1g018958m) also exhibited generally higher expression levels in HLB-tolerant citrus varieties than susceptible varieties.

### Example 10

### Data Availability

The mass spectrometry data generated in this study has been deposited in the PRIDE Archive (http www address ebi.ac.uk/pride/archive) under accession number PXD008366.

TABLE 4
Primers used in this study.

| | | | | |
|---|---|---|---|---|
| | | SEQ | | |
| Primer | | ID | Gene | Accession |
| Name | Sequence 5'-3' | NO: | Name | Number |

qRT-PCR primers

| | | | | |
|---|---|---|---|---|
| CsPR1F | AAAGTTGTTCAAACTTTTTGTCCTT | 6 | Pathogenesis | EF010853.1 |
| CsPR1R | ACATGATCAATAGTAGGGATGTTAGC | 7 | Related | |
| | | | gene | |
| COXF | GTATGCCACGTCGCATTCCAGA | 8 | Cytochrome | KF933043.1 |
| COXR | GCCAAAACTGCTAAGGGCATTC | 9 | Oxidase | |
| | | | subunit 1 | |
| CcRD21aF | GACCGTCTCCTCCATCTCCAAC | 10 | Granulin | XM_006473212 |
| CcRD21aR | GTCCTTGCTCATCAAGCAGGTC | 11 | repeat | |
| | | | cysteine | |
| | | | protease | |
| | | | family | |
| | | | protein | |
| CsSAG12- | AAGGTCAATGTGGAGACTGTTG | 12 | ervatamin- | XM_006495158 |
| 1F | | | B-like | |
| CsSAG12- | TTGAGCAGTCTAGTATTTGC | 13 | | |
| 1R | | | | |
| CsSAG12- | AGGCCAATGTGGATCCTGTTGG | 14 | Cysteine | orange1.1g019063m |
| 4F | | | proteinases | |
| CsSAG12- | ACCAACTGCTGCTCCGAAAG | 15 | superfamily | |
| 4R | | | protein | |
| CsAALPF | ATGTGAACCATGCCGTCGTTG | 16 | Cysteine | orange1.1g036910m |
| | | | proteinases | |
| CsAALPR | GTTGCAATACCACACATGTT | 17 | superfamily | |
| | | | protein | |
| CsXBCP3F | AGTGACTGAGGTCAAAGATCAAG | 18 | Xylem | orange1.1g014761m |
| | | | bark | |
| CsXBCP3R | GACGAGAGAGCCAGTAACAATC | 19 | cysteine | |
| | | | peptidase | |

SDE1 and PLCP cloning primers- yeast-two-hybrid

| | | | | |
|---|---|---|---|---|
| 5315-no- | TGCCATGGAGGGCAGTAGTTT | 20 | SDE1 | CLIBASIA_05315 |
| SP-F | | | | |
| 5315-R | TGGGATCCGTAGACTGCTCCA | 21 | | |
| SAG12-1- | CCATCGATCATCTAAAATGGTGTCGGTC | 22 | CsSAG12-1 | XM_006495158 |
| full- | GC | | | |
| length-F | | | | |
| SAG12-1- | CCATCGATTTCGTGCTTCATATACAGGA | 23 | | |
| cys-F | TACAG | | | |
| SAG12-1- | CGGGATCC | 24 | | |
| R | TCATGCAACTGGGTAGGAAGG | | | |
| SAG12-2- | GGAATTCCATATGCGGTCGATGCATGA | 25 | CsSAG12-2 | XM_006470229 |
| full- | ACCG | | | |
| length-F | | | | |
| SAG12-2- | GGGAATTCCATATGCGTGCTTTATATAC | 26 | | |
| cys-F | AGGA | | | |
| SAG12-2- | TCCCCCGGGTCCATTGCAACTGGATA | 27 | | |
| R | | | | |
| AALP- | GGAATTCCATATGTCAGCATCAAGCTTC | 28 | CsAALP | XM_006474664 |
| full- | GAC | | | |
| length-F | | | | |
| AALP- | GGAATTCCATATGCAAAGGCACAGGTT | 29 | | |
| cys-F | GGGAGC | | | |
| AALP-R | CGGGATCCCTAAGCCACAACTGGGTAT | 30 | | |
| | G | | | |
| RD21a- | CCATCGATCCTTGGACATGTCGATTGTA | 31 | CsRD21a | XM_006473212 |
| full- | TC | | | |
| length-F | | | | |
| RD21a- | CCATCGATACCGGTCCATGCATCTGG | 32 | | |
| cys-F | | | | |
| RD21a-R | CGGGATCCTTAAGCACTGCTACTTCCAC | 33 | | |
| | C | | | |
| XCBP3- | TCCCCCGGGTTCAGACATCAATGAACTC | 34 | CsXCBP3 | orange1.1g012960m |
| full- | TTTGA | | | |
| length-F | | | | |
| XCBP3-R | CGGGATCCTTATACAAACCAAGCATCA | 35 | | |
| | ATGAAGG | | | |
| RD19- | GGAATTCCATATG | 36 | CsRD19 | orange1.1g017548m |
| full- | GTCAACGACGACGATGCTAT | | | |
| length-F | | | | |
| RD19-R | TCCCCCGGGCTAGCTTGAGGTTGTAT | 37 | | |
| CTB-full- | GGAATTCCATATG | 38 | CsCTB | orange1.1g018568m |
| length-F | GAGGGAGTGGTTTCCAAGCT | | | |
| CTB-R | TCCCCCGGGTCAAGCTGAAGCATCCTCA | 39 | | |
| | A | | | |
| SAG12- | CGCCATATGGTAAGCCGCCAGTCATCA | 40 | CsSAG12-3 | orange1.1g018958m |
| 63-cys-F | CG | | | |
| SAG12- | CCGCTCGAGTCATATTGCAACTGGATAG | 41 | | |
| 63-R | G | | | |
| SAG12- | CGCCATATGTCCACAACAGCATCCTTCA | 42 | CcSAG12-1 | Ciclev10005334 |
| 34-cys-F | AG | | | |
| SAG12- | CCGCTCGAGTCATGCAACTGGGTAGGA | 43 | | |
| 34-R | AG | | | |
| SAG12- | CGCCATATGCATCGATCCACAACATCAT | 44 | CsSAG12-4 | orange1.1g019063m |
| 58-cys-F | CC | | | |
| SAG12- | CCGCTCGAGTCATGCAAGTGGATAGGA | 45 | | |
| 58-R | AG | | | |

SDE1 and PLCP cloning primers- pull-down

| | | | | |
|---|---|---|---|---|
| 5315-no- | ATCGGGATCCGGCAGTAGTTTTGGTTGT | 46 | SDE1 | CLIBASIA_05315 |
| SP-F | TGT | | | |
| 5315-R | CCGACGTCGACTCA | 47 | | |
| | AGACTGCTCCAACATTTTTCTATGG | | | |
| rcr3-cys- | TCCCCCGGGGGATGTTGCTGGGCGTTTT | 48 | SlRCR3 | AFP73354.1 |
| F | C | | | |
| rcr3-R | CCGCTCGAGCTATGCTATGTTTGGATAA | 49 | | |
| | GAAGAC | | | |
| SAG12-1- | GCATCCTTCAAGTATCAAAACCTG | 50 | CsSAG12-1 | XM_006495158 |
| cys-F | | | | |
| SAG12-1- | TCATGCAACTGGGTAGGAAG | 51 | | |
| R | | | | |
| SAG12-2- | TCCACCTTCAAGTACCAAAACG | 52 | CsSAG12-2 | XM_006470229 |
| cys-F | | | | |
| SAG12-2- | TCACATTGCAACTGGATAGGAAG | 53 | | |
| R | | | | |
| AALP- | AAAGGAAATCACAAGCTTACTG | 54 | CsAALP | XM_006474664 |
| cys-F | | | | |
| AALP-R | CTAAGCCACAACTGGGTATGATG | 55 | | |
| RD21a- | GATCGTTATCTGCCACGTGTC | 56 | CsRD21a | XM_006473212 |
| cys-F | | | | |
| RD21a-R | TTAAGCACTGCTACTTCCACCTTG | 57 | | |
| XCBP3- | TCTGTTCAATCGCCCGGTAC | 58 | CsXCBP3 | orange1.1g012960m |
| cys-F | | | | |
| XCBP3-R | TTATACAAACCAAGCATCAATGAAGG | 59 | | |
| RD19- | CAAAAGGCTCCTATCCTCCCTAC | 60 | CsRD19 | orange1.1g017548m |
| cys-F | | | | |
| RD19-R | CTAGCTTGAGGTTGTATGGATAGC | 61 | | |
| CTB-cys- | CCTGTTAAAACTCATGACAAATCC | 62 | CsCTB | orange1.1g018568m |
| F | | | | |
| CTB-R | TCAAGCTGAAGCATCCTCAAAC | 63 | | |

### REFERENCES

The following references are cited in the disclosure by reference number:
1 Bové, J. M. Huanglongbing: a destructive, newly-emerging, century-old disease of citrus. Journal of Plant Pathology 88, 7-37 (2006).
2 da Graça, J. V. et al. Huanglongbing: An overview of a complex pathosystem ravaging the world's citrus. Journal of Integrative Plant Biology 58, 373-387 (2016).
3 Gottwald, T. R. Current epidemiological understanding of citrus Huanglongbing. Annual Review of Phytopathology 48, 119-139 (2010).
4 Wang, N. & Trivedi, P. Citrus Huanglongbing: a newly relevant disease presents unprecedented challenges. Phytopathology 103, 652-665 (2013).
5 Wang, N. et al. The Candidatus Liberibacter—host interface: insights into pathogenesis mechanisms and disease control. Annual Review of Phytopathology 55, 451-482 (2017).
6 Spreen, T. H. & Hodges, A. W. Economic impacts of citrus greening (HLB) in Florida, 2006/07-2010/11, http://ufdc.ufl.edu/IR00005615/00001 (2012).
7 Spreen, T. H. et. al. An economic assesment of the impact of Huanglongbing on citrus tree 640 plantings in Florida. HortScience 49, 1052-1055 (2014).
8 Toruño, T. Y., Stergiopoulos, I. & Coaker, G. Plant-pathogen effectors: cellular probes interfering with plant defenses in spatial and temporal manners. Annual Review of Phytopathology 54, 419-441 (2016).
9 Dodds, P. N. & Rathj en, J. P. Plant immunity: towards an integrated view of plant—pathogen interactions. Nature Reviews Genetics 11, 539-548 (2010).
10 Feng, F. & Zhou, J.-M. Plant—bacterial pathogen interactions mediated by type III effectors. Current Opinion in Plant Biology 15, 469-476 (2012).
11 Sugio, A. et al. Diverse targets of phytoplasma effectors: from plant development to defense against insects. Annual Review of Phytopathology 49, 175-195 (2011).
12 MacLean, A. M. et al. Phytoplasma effector SAP54 induces indeterminate leaf-like flower development in Arabidopsis plants. Plant Physiology 157, 831-841 (2011).
13 Hoshi, A. et al. A unique virulence factor for proliferation and dwarfism in plants identified from a phytopathogenic bacterium. Proceedings of the National Academy of Sciences 106, 6416-6421 (2009).
14 Yan, Q. et al. Global gene expression changes in Candidatus Liberibacter asiaticus during the transmission in distinct hosts between plant and insect. Molecular Plant Pathology 14, 391-404 (2013).
15 Prasad, S., Xu, J., Zhang, Y. & Wang, N. SEC-translocon dependent extracytoplasmic proteins of Candidatus Liberibacter asiaticus. Frontiers in Microbiology 7, 1989 (2016).
16 Pagliaccia, D. et al. A pathogen secreted protein as a detection marker for citrus Huanglongbing. Frontiers in Microbiology 8, 2041 (2017).
17 Pitino, M., Armstrong, C. M., Cano, L. M. & Duan, Y. Transient expression of Candidatus Liberibacter asiaticus effector induces cell death in Nicotiana benthamiana. Frontiers in Plant Science 7, 982 (2016).
18 Jashni, M., Mehrabi, R., Collemare, J., Mesarich, C. & de Wit, P. The battle in the apoplast: further insights into the roles of proteases and their inhibitors in plant-pathogen interactions. Frontiers in Plant Science 6, 584 (2015).
19 Misas-Villamil, J. C., Hoorn, R. A. & Doehlemann, G. Papain-like cysteine proteases as hubs in plant immunity. New Phytologist 212, 902-907 (2016).
20 Ilyas, M. et al. Functional divergence of two secreted immune proteases of tomato. Current Biology 25, 2300-2306 (2015).
21 Lozano-Torres, J. L. et al. Dual disease resistance mediated by the immune receptor Cf-2 in tomato requires a common virulence target of a fungus and a nematode. Proceedings of the National Academy of Sciences 109, 10119-10124 (2012).
22 Richau, K. H. et al. Subclassification and biochemical analysis of plant papain-like cysteine proteases displays subfamily-specific characteristics. Plant Physiology 158, 1583-1599 (2012).
23 Than, M. E. et al. The 2.0 Å crystal structure and substrate specificity of the KDEL-tailed (SEQ ID NO: 64) cysteine endopeptidase functioning in programmed cell death of Ricinus communis endosperm. J Mol Biol 336, 1103-1116 (2004).
24 Powers, J. C., Asgian, J. L., Ekici, Ö. D. & James, K. E. Irreversible inhibitors of serine, cysteine, and threonine proteases. Chemical Reviews 102, 4639-4750 (2002).
25 Rooney, H. C. et al. Cladosporium Avr2 inhibits tomato Rcr3 protease required for Cf-2-dependent disease resistance. Science 308, 1783-1786 (2005).
26 Chen, Z., Zheng, Z., Huang, J., Lai, Z. & Fan, B. Biosynthesis of salicylic acid in plants. Plant Signaling & Behavior 4, 493-496 (2009).
27 Piazza, A. et al. The dual nature of trehalose in citrus canker disease: a virulence factor for Xanthomonas citri subsp. citri and a trigger for plant defence responses. Journal of Experimental Botany 66, 2795-2811 (2015).
28 Martinelli, F. et al. Transcriptome profiling of citrus fruit response to Huanglongbing disease. PLoS One 7, e38039 (2012).
29 Kim, J.-S., Sagaram, U. S., Burns, J. K., Li, J.-L. & Wang, N. Response of sweet orange (Citrus sinensis) to ‘Candidatus Liberibacter asiaticus’ infection: microscopy and microarray analyses. Phytopathology 99, 50-57 (2009).
30 Ahmed, S. U. et al. The plant vacuolar sorting receptor AtELP is involved in transport of NH2-terminal propeptide-containing vacuolar proteins in Arabidopsis thaliana. The Journal of Cell Biology 149, 1335-1344 (2000).
31 Sueldo, D. et al. Dynamic hydrolase activities precede hypersensitive tissue collapse in tomato seedlings. New Phytologist 203, 913-925 (2014).
32 Shindo, T. et al. Screen of non-annotated small secreted proteins of Pseudomonas syringae reveals a virulence factor that inhibits tomato immune proteases. PLoS Pathogens 12, e1005874 (2016).
33 Jiang, S. et al. Bacterial effector activates jasmonate signaling by directly targeting JAZ transcriptional repressors. PLoS Pathogens 9, e1003715 (2013).
34 Kaschani, F. et al. An effector-targeted protease contributes to defense against Phytophthora infestans and is under diversifying selection in natural hosts. Plant Physiology 154, 1794-1804 (2010).
35 Shindo, T., Misas-Villamil, J. C., Horger, A. C., Song, J. & van der Hoorn, R. A. A role in immunity for Arabidopsis cysteine protease RD21, the ortholog of the tomato immune protease C14. PLoS One 7, e29317 (2012).
36 Gumtow, R., Wu, D., Uchida, J. & Tian, M. A Phytophthora palmivora extracellular cystatin-like protease inhibitor targets papain to contribute to virulence on papaya. Molecular Plant-Microbe Interactions 31, 363-373 (2017).
37 Tian, M. et al. A Phytophthora infestans cystatin-like protein targets a novel tomato papain-like apoplastic protease. Plant Physiology 143, 364-377 (2007).
38 Dong, S. et al. Effector specialization in a lineage of the Irish potato famine pathogen. Science 343, 552-555 (2014).
39 Bozkurt, T. O. et al. Phytophthora infestans effector AVRb1b2 prevents secretion of a plant immune protease at the haustorial interface. Proceedings of the National Academy of Sciences 108, 20832-20837 (2011).
40 van Esse, H. P. et al. The Cladosporium fulvum virulence protein Avr2 inhibits host proteases required for basal defense. Plant Cell 20, 1948-1963 (2008).
41 Mueller, A. N., Ziemann, S., Treitschke, S., Assmann, D. & Doehlemann, G. Compatibility in the Ustilago maydis-maize interaction requires inhibition of host cysteine proteases by the fungal effector Pit2. PLoS Pathogens 9, e1003177 (2013).
42 Bove, J. & Gamier, M. Phloem- and xylem-restricted plant pathogenic bacteria. Plant Science 164, 423-438 (2003).
43 Lopez-Cobollo, R. M., Filippis, I., Bennett, M. H. & Turnbull, C. G. Comparative proteomics of cucurbit phloem indicates both unique and shared sets of proteins. The Plant Journal 88, 633-647 (2016).
44 Hu, C. et al. Proteomics and metabolomics analyses reveal the cucurbit sieve tube system as a complex metabolic space. The Plant Journal 87, 442-454 (2016).
45 Orbovié, V. & Grosser, J. W. in Agrobacterium Protocols: Volume 2 (ed Kan Wang) 245-257 (Springer N.Y., 2015).
46 Schultz, J., Milpetz, F., Bork, P. & Ponting, C. P. SMART, a simple modular architecture research tool: identification of signaling domains. Proceedings of the National Academy of Sciences 95, 5857-5864 (1998).
47 Letunic, I., Doerks, T. & Bork, P. SMART: recent updates, new developments and status in 2015. Nucleic Acids Research 43, D257-D260 (2014).
48 Edgar, R. C. MUSCLE: a multiple sequence alignment method with reduced time and space complexity. BMC Bioinformatics 5, 113 (2004).
49 Tamura, K., Stecher, G., Peterson, D., Filipski, A. & Kumar, S. MEGA6: Molecular Evolutionary Genetics Analysis version 6.0. Molecular Biology and Evolution 30, 2725-2729 (2013).
50 Laemmli, U. K. Cleavage of structural proteins during the assembly of the head of bacteriophage T4. Nature 227, 680-685 (1970).
51 Morgan, J. K. et al. Improved real-time PCR detection of Candidatus ‘Liberibacter asiaticus’ from citrus and psyllid hosts by targeting the intragenic tandem-repeats of its prophage genes. Molecular and Cellular Probes 26, 90-98 (2012).
52 Yadeta, K. A. et al. An extracellular cysteine-rich protein kinase associates with a membrane immune complex and is required for cell death. Plant Physiology, pp. 01404 752 (2016).
53 Cox, J. & Mann, M. MaxQuant enables high peptide identification rates, individualized ppb-range mass accuracies and proteome-wide protein quantification. Nature Biotechnology 26, 1367-1372 (2008).
54 Cox, J. et al. Andromeda: a peptide search engine integrated into the MaxQuant environment. Journal of Proteome Research 10, 1794-1805 (2011).
55 Cox, J. et al. Accurate proteome-wide label-free quantification by delayed normalization and maximal peptide ratio extraction, termed MaxLFQ. Molecular & Cellular Proteomics 9, 2513-26 (2014).
56 Vizcaino, J. A. et al. The PRoteomics IDEntifications (PRIDE) database and associated tools: status in 2013. Nucleic Acids Research 41, D1063-D1069 (2013).
57 Tyanova, S. et al. The Perseus computational platform for comprehensive analysis of (prote)omics data. Nat Methods 13, 731-740 (2016).
58 Pieper, U. et al. MODBASE, a database of annotated comparative protein structure models, and associated resources. Nucleic Acids Research 32, D217-D222 (2004).
59 Pettersen, E. F. et al. UCSF Chimera—a visualization system for exploratory research and analysis. Journal of Computational Chemistry 25, 1605-1612 (2004).
60 Morgan, R. L. et al. Catalytic domain of the diversified Pseudomonas syringae type III effector HopZ1 determines the allelic specificity in plant hosts. Molecular Microbiology 2, 437-55 (2010).
61 Paszkowski, J. et al. Direct gene transfer to plants. EMBO J 3, 2717-2722 (1984).
62 Fromm, M. et al. Expression of genes transferred into monocot and dicot plant cells by electroporation. Proc. Natl. Acad. Sci. USA 82 5824-5828 (1985).
63 Klein, T. M. et al. High-Velocity Microprojectiles for Delivering Nucleic Acids into Living Cells. Nature 327, 70-73 (1987).
64 Horsch, R. B. et al., Inheritance of Functional Foreign Genes in Plants. Science 223, 496-498 (1984).
65 Fraley, R. T. et al., Expression of bacterial genes in plant cells. Proc. Natl. Acad. Sci. USA 80, 4803-4807(1983).
66 Evans et al., Protoplasts Isolation and Culture, Handbook of Plant Cell Culture, pp. 124-176, MacMillan Publishing Company, New York (1983).
67 Binding, Regeneration of Plants, Plant Protoplasts, pp. 21-73, CRC Press, Boca Raton (1985).
68 Klee, H. et al. Agrobacterium-mediated plant transformation and its further applications to plant biology. Ann. Rev. of Plant Phys. 38, 467-486 (1987).
69 Jinek et al., A Programmable Dual-RNA—Guided DNA Endonuclease in Adaptive Bacterial Immunity. Science 337, 816-821 (2012).
70 Hsu, P. D. et al., Development and applications of CRISPR-Cas9 for genome engineering. Cell 157, 1262-1278 (2014).
71 Li, J. F. et al., Multiplex and homologous recombination-mediated genome editing in Arabidopsis and Nicotiana benthamiana using guide RNA and Cas9. Nat Biotechnol 31, 688-691 (2013).
72 Shan, Q. et al. Targeted genome modification of crop plants using a CRISPR-Cas system. Nat Biotechnol 31, 686-688 (2013).
73 Chylinksi, K. et al., The tracrRNA and Cas9 families of type II CRISPR-Cas immunity systems. RNA Biol. 10, 726-737 (2010).
74 Hou, Z. et al., Efficient genome engineering in human pluripotent stem cells using Cas9 from Neisseria meningitidis PNAS 110, 15644-15649 (2013).
75 Makarova, K. S., et al., Evolution and classification of the CRISPR-Cas systems. Nat Rev Microbiol 9, 467-477 (2011).
76 Sampson et al., A CRISPR/Cas system mediates bacterial innate immune evasion and virulence. Nature 497, 254-257 (2013).
77 Myers, E. W. and Miller, W., Optimal alignments in linear space., Comput. Applic. Biosci. 4, 11-17 (1988).
78 Smith, T. F. and M. S. Waterman, Comparison of Biosequences. Adv. Appl. Math. 2, 482-489 (1981).
79 Needleman, S. B., Wunsch, C. D., A general method applicable to the search for similarities in the amino acid sequence of two proteins. J. Mol. Biol. 48, 443-453 (1970).
80 Pearson, W. R., and Lipman, D. J., Improved tools for biological sequence comparison. Natl. Acad. Sci. USA 85, 2444-2448 (1988).
81 Altschul, S. F., et al., Basic local alignment search tool. J. Mol. Bio. 215, 403-410 (1990).
82 Altschul, S. F., et al., Gapped BLAST and PSI-BLAST: a new generation of protein database search programs. Nucleic Acids Research 25, 3389-3402 (1997).
83 Henikoff & Henikoff, Amino acid substitution matrices from protein blocks. Proc. Natl. Acad Sci. USA 89,10915-10919 (1992).
84 Karlin & Altschul, Applications and statistics for multiple high-scoring segments in molecular sequences. Proc. Natl. Acad. Sci. USA 90, 5873-5787 (1993).

All publications, including accession number, patents and patent applications cited in this specification are herein incorporated by reference as if each individual publication or patent application were specifically and individually indicated to be incorporated by reference. All accession numbers are incorporated by reference for each individual sequence provided under the accession number.

Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

Illustrative PLCP polypeptide sequences
SEQ ID NO: 1 CsSAG12_1 Full-length polypeptide
sequence encoded by gene XM_006495158
(NCBI Accession No.).
The signal peptide sequence is underlined
MEKSFDIIALSMIILVTYSSKMVSVAGRSLHEPSIVEKHEKWMAEHGRTY
KDDLEKEKRFKIFKENLEYIEKANEEANRTYKLGTNEFSDLTNEEFRASY
TGYRVPSQSSSSRQSTTASFKYQNLTDVPTSMDWREKGAVTPIKNQGQCG
DCWAFSAVAAVEGVTEISSGNLIPLSEQQILDCSTDGNRGCGGGWMDNAF
KYIIQNQGIASEADYPYKEVQGTCEDAQVKVAAKISNFEDVKPNDEQALL
QAVAMQPVSICIEGSGPDFQSYKGGIFNGGCGTQCSHAVAIVGFGATEDG
MKYWLIKNSWGESWGEAGYMRILRDVEAPEGLCGIATKPSYPVA*
SEQ ID NO: 2. CsSAG12_3 Full length sequence
polypeptide sequence encoded by gene
orange1.1g018958m The signal peptide sequence is
underlined.
MVLIFERSGSFKINTTPMFIIITLLVSCASQVVSSRSTHEQSVVEIHEKW
MAQHGRSYKDELEKEMRLKIFKENLEYIEKANKEGNRTYKLGTNQFSDLT
NDEFRALYTGYKMPSPSHRSTTSSTFKYQNLSMTDVPTSLDWRDKGAVTP
IKNQKECGCCWAFAAVAAVEGITKIRSGNLIQLSEQQLLDCSTNGNNGCL
GGSREKAFAYIIQNQGIATEDEYPYQAVPGTCSAAQKPAAAKISNYEEVS
GDEQALLKAVSMQPVSIAIAAYSTEFQSYKEGIFNGVCGTQLDHAVPTIV
GFGTTEDGANYWLIKNSWGNTWGDAGYMKIVRDEGLCGIGTRSSYPLA*

What is claimed is:

1. A genetically modified citrus plant or plant cell comprising a papain-like cysteine protease (PLCP) polypeptide, wherein the PLCP polypeptide is heterologously expressed or is a mutant PLCP that has reduced binding to Candidatus Liberibacter asiaticus (CLas) effector SDE1 compared to a corresponding wildtype PLCP protein.

2. The genetically modified citrus plant or plant cell of claim 1, wherein the PLCP polypeptide is heterologously expressed.

3. The genetically modified citrus plant or plant cell of claim 2, wherein the citrus plant is a transgenic citrus plant comprising a heterologous expression cassette comprising a promoter operably linked to a nucleic acid sequence encoding the PLCP polypeptide.

4. The genetically modified citrus plant or plant cell of claim 2, wherein the PLCP is encoded from an endogenous coding sequence that is linked to a modified PLCP promoter sequence comprising at least one nucleotide alteration compared to the native PLCP promoter sequence.

5. The genetically modified citrus plant or plant cell of claim 1, wherein the PLCP polypeptide is a mutant PLCP that has reduced binding to Candidatus Liberibacter asiaticus (CLas) effector SDE1 compared to a corresponding wildtype PLCP protein, wherein the mutant PLCP has from one to ten amino acid changes compared to the wildtype PLCP protein.

6. The genetically modified citrus plant or plant cell claim 1, wherein the citrus plant has enhanced resistance to infection or damage by CLas compared to a control plant that lacks the heterologously expressed PLCP polypeptide or mutant PLCP.

7. The genetically modified citrus plant or plant cell of claim 6, wherein the PLCP polypeptide is from a PLCP subfamily of SAG12, THL1, CEP1, XCP1, XBCP3, RD21a, RD19, AALP and CTB.

8. The genetically modified citrus plant or plant cell of claim 6, wherein the PLCP polypeptide is at least 95% identical to a native PLCP polypeptide listed in FIG. 1.

9. The genetically modified citrus plant or plant cell claim 1, wherein the mature region of the PLCP polypeptide has at least 70% identity to amino acids 21-344 of SEQ ID NO:1 or amino acids 36-348 SEQ ID NO:2.

10. The genetically modified citrus plant or plant cell claim 9, wherein the mature region of the PLCP polypeptide has at least 90% identity to amino acids 21-344 of SEQ ID NO:1 or amino acids 36-348 SEQ ID NO:2. 11 . The genetically modified citrus plant or plant cell claim 1, wherein the citrus plant is selected from the group consisting Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus sinensis, and Citrus tangerine, and Citrus clementina.

12. A method of making citrus plant has enhanced resistance to infection or damage by CLas, the method comprising, introducing into a citrus plant an alteration in a promoter operably linked to a nucleic acid sequence encoding a native papain-like cysteine protease (PLCP) polypeptide, wherein the alteration results in increased expression the PLCP polypeptide compared to the native PLCP polypeptide.

13. The method of claim 12, wherein the promoter is a native promoter and the introducing comprises introducing a targeted nuclease that cleaves a target region in that native promoter and a heterologous nucleic acid sequence is introduced into the native promoter thereby introducing the alteration into the native promoter.

14. The method of claim 13, wherein the nuclease is an RNA-guided nuclease.

15. The method of claim 14, wherein the RNA-guided nuclease is a Cpf1 nuclease or a Cas9 nuclease and the method further comprises introducing into the cell a guide RNA that specifically hybridizes to the target region.

16. The method of claim 12, wherein the mature region of the native PLCP polypeptide has at least 70% identity to the mature region of SEQ ID NO:1 or the mature region of SEQ ID NO:2.

17. The method of claim 12, wherein the citrus plant is selected from the group consisting Citrus maxima, Citrus medica, Citrus micrantha, Citrus reticulata, Citrus aurantiifolia, Citrus aurantium, Citrus latifolia, Citrus limon, Citrus limonia, Citrus paradisi, Citrus sinensis, and Citrus tangerine, and Citrus clementina.

18. A citrus plant cell comprising a promoter operably linked to a polynucleotide encoding the mutant PLCP of claim 5.


SEQUENCE LISTING (64 sequences)

SEQ ID NO: 1 - protein, 344 aa, Citrus sinensis
MEKSFDIIALSMIILVTYSSKMVSVAGRSLHEPSIVEKHEKWMAEHGRTYKDDLEKEKRFKIFKENLEYIEKANEEANRTYKLGTNEFSDLTNEEFRASYTGYRVPSQSSSSRQSTTASFKYQNLTDVPTSMDWREKGAVTPIKNQGQCGDCWAFSAVAAVEGVTEISSGNLIPLSEQQILDCSTDGNRGCGGGWMDNAFKYIIQNQGIASEADYPYKEVQGTCEDAQVKVAAKISNFEDVKPNDEQALLQAVAMQPVSICIEGSGPDFQSYKGGIFNGGCGTQCSHAVAIVGFGATEDGMKYWLIKNSWGESWGEAGYMRILRDVEAPEGLCGIATKPSYPVA

SEQ ID NO: 2 - protein, 348 aa, Citrus sinensis
MVLIFERSGSFKINTTPMFIIITLLVSCASQVVSSRSTHEQSVVEIHEKWMAQHGRSYKDELEKEMRLKIFKENLEYIEKANKEGNRTYKLGTNQFSDLTNDEFRALYTGYKMPSPSHRSTTSSTFKYQNLSMTDVPTSLDWRDKGAVTPIKNQKECGCCWAFAAVAAVEGITKIRSGNLIQLSEQQLLDCSTNGNNGCLGGSREKAFAYIIQNQGIATEDEYPYQAVPGTCSAAQKPAAAKISNYEEVPSGDEQALLKAVSMQPVSIAIAAYSTEFQSYKEGIFNGVCGTQLDHAVTIVGFGTTEDGANYWLIKNSWGNTWGDAGYMKIVRDEGLCGIGTRSSYPLA

SEQ ID NO: 3 - protein, 6 aa, Artificial Sequence
  Description of Artificial Sequence Synthetic 6xHis tag
HHHHHH

SEQ ID NO: 4 - DNA, 39 bp, Artificial Sequence
  Description of Artificial Sequence Synthetic primer
gtaaagcttcaccatcaccatcaccatgccaagaaatta

SEQ ID NO: 5 - DNA, 23 bp, Artificial Sequence
  Description of Artificial Sequence Synthetic primer
ctgagatctcaaccacaaagtcc

SEQ ID NO: 6 - DNA, 25 bp, Artificial Sequence
  Description of Artificial Sequence Synthetic primer
aaagttgttcaaactttttgtcctt

SEQ ID NO: 7 - DNA, 26 bp, Artificial Sequence
  Description of Artificial Sequence Synthetic primer
acatgatcaatagtagggatgttagc

SEQ ID NO: 8 - DNA, 22 bp, Artificial Sequence
  Description of Artificial Sequence Synthetic primer
gtatgccacgtcgcattccaga

SEQ ID NO: 9 - DNA, 22 bp, Artificial Sequence
  Description of Artificial Sequence Synthetic primer
gccaaaactgctaagggcattc

SEQ ID NO: 10 - DNA, 22 bp, Artificial Sequence
  Description of Artificial Sequence Synthetic primer
gaccgtctcctccatctccaac

SEQ ID NO: 11 - DNA, 22 bp, Artificial Sequence
  Description of Artificial Sequence Synthetic primer
gtccttgctcatcaagcaggtc

SEQ ID NO: 12 - DNA, 22 bp, Artificial Sequence
  Description of Artificial Sequence Synthetic primer
aaggtcaatgtggagactgttg

SEQ ID NO: 13 - DNA, 20 bp, Artificial Sequence
  Description of Artificial Sequence Synthetic primer
ttgagcagtctagtatttgc

SEQ ID NO: 14 - DNA, 22 bp, Artificial Sequence
  Description of Artificial Sequence Synthetic primer
aggccaatgtggatcctgttgg

SEQ ID NO: 15 - DNA, 20 bp, Artificial Sequence
  Description of Artificial Sequence Synthetic primer
accaactgctgctccgaaag

SEQ ID NO: 16 - DNA, 21 bp, Artificial Sequence
  Description of Artificial Sequence Synthetic primer
atgtgaaccatgccgtcgttg

SEQ ID NO: 17 - DNA, 20 bp, Artificial Sequence
  Description of Artificial Sequence Synthetic primer
gttgcaataccacacatgtt

SEQ ID NO: 18 - DNA, 23 bp, Artificial Sequence
  Description of Artificial Sequence Synthetic primer
agtgactgaggtcaaagatcaag

SEQ ID NO: 19 - DNA, 22 bp, Artificial Sequence
  Description of Artificial Sequence Synthetic primer
gacgagagagccagtaacaatc

SEQ ID NO: 20 - DNA, 21 bp, Artificial Sequence
  Description of Artificial Sequence Synthetic primer
tgccatggagggcagtagttt

SEQ ID NO: 21 - DNA, 21 bp, Artificial Sequence
  Description of Artificial Sequence Synthetic primer
tgggatccgtagactgctcca

SEQ ID NO: 22 - DNA, 30 bp, Artificial Sequence
  Description of Artificial Sequence Synthetic primer
ccatcgatcatctaaaatggtgtcggtcgc

SEQ ID NO: 23 - DNA, 33 bp, Artificial Sequence
  Description of Artificial Sequence Synthetic primer
ccatcgatttcgtgcttcatatacaggatacag

SEQ ID NO: 24 - DNA, 29 bp, Artificial Sequence
  Description of Artificial Sequence Synthetic primer
cgggatcctcatgcaactgggtaggaagg

SEQ ID NO: 25 - DNA, 31 bp, Artificial Sequence
  Description of Artificial Sequence Synthetic primer
ggaattccatatgcggtcgatgcatgaaccg

SEQ ID NO: 26 - DNA, 32 bp, Artificial Sequence
  Description of Artificial Sequence Synthetic primer
gggaattccatatgcgtgctttatatacagga

SEQ ID NO: 27 - DNA, 26 bp, Artificial Sequence
  Description of Artificial Sequence Synthetic primer
tcccccgggtccattgcaactggata

SEQ ID NO: 28 - DNA, 31 bp, Artificial Sequence
  Description of Artificial Sequence Synthetic primer
ggaattccatatgtcagcatcaagcttcgac

SEQ ID NO: 29 - DNA, 33 bp, Artificial Sequence
  Description of Artificial Sequence Synthetic primer
ggaattccatatgcaaaggcacaggttgggagc

SEQ ID NO: 30 - DNA, 28 bp, Artificial Sequence
  Description of Artificial Sequence Synthetic primer
cgggatccctaagccacaactgggtatg

SEQ ID NO: 31 - DNA, 30 bp, Artificial Sequence
  Description of Artificial Sequence Synthetic primer
ccatcgatccttggacatgtcgattgtatc

SEQ ID NO: 32 - DNA, 26 bp, Artificial Sequence
  Description of Artificial Sequence Synthetic primer
ccatcgataccggtccatgcatctgg

SEQ ID NO: 33 - DNA, 29 bp, Artificial Sequence
  Description of Artificial Sequence Synthetic primer
cgggatccttaagcactgctacttccacc

SEQ ID NO: 34 - DNA, 33 bp, Artificial Sequence
  Description of Artificial Sequence Synthetic primer
tcccccgggttcagacatcaatgaactctttga

SEQ ID NO: 35 - DNA, 34 bp, Artificial Sequence
  Description of Artificial Sequence Synthetic primer
cgggatccttatacaaaccaagcatcaatgaagg

SEQ ID NO: 36 - DNA, 33 bp, Artificial Sequence
  Description of Artificial Sequence Synthetic primer
ggaattccatatggtcaacgacgacgatgctat

SEQ ID NO: 37 - DNA, 26 bp, Artificial Sequence
  Description of Artificial Sequence Synthetic primer
tcccccgggctagcttgaggttgtat

SEQ ID NO: 38 - DNA, 33 bp, Artificial Sequence
  Description of Artificial Sequence Synthetic primer
ggaattccatatggagggagtggtttccaagct

SEQ ID NO: 39 - DNA, 29 bp, Artificial Sequence
  Description of Artificial Sequence Synthetic primer
tcccccgggtcaagctgaagcatcctcaa

SEQ ID NO: 40 - DNA, 29 bp, Artificial Sequence
  Description of Artificial Sequence Synthetic primer
cgccatatggtaagccgccagtcatcacg

SEQ ID NO: 41 - DNA, 29 bp, Artificial Sequence
  Description of Artificial Sequence Synthetic primer
ccgctcgagtcatattgcaactggatagg

SEQ ID NO: 42 - DNA, 30 bp, Artificial Sequence
  Description of Artificial Sequence Synthetic primer
cgccatatgtccacaacagcatccttcaag

SEQ ID NO: 43 - DNA, 29 bp, Artificial Sequence
  Description of Artificial Sequence Synthetic primer
ccgctcgagtcatgcaactgggtaggaag

SEQ ID NO: 44 - DNA, 30 bp, Artificial Sequence
  Description of Artificial Sequence Synthetic primer
cgccatatgcatcgatccacaacatcatcc

SEQ ID NO: 45 - DNA, 29 bp, Artificial Sequence
  Description of Artificial Sequence Synthetic primer
ccgctcgagtcatgcaagtggataggaag

SEQ ID NO: 46 - DNA, 31 bp, Artificial Sequence
  Description of Artificial Sequence Synthetic primer
atcgggatccggcagtagttttggttgttgt

SEQ ID NO: 47 - DNA, 39 bp, Artificial Sequence
  Description of Artificial Sequence Synthetic primer
ccgacgtcgactcaagactgctccaacatttttctatgg

SEQ ID NO: 48 - DNA, 29 bp, Artificial Sequence
  Description of Artificial Sequence Synthetic primer
tcccccgggggatgttgctgggcgttttc

SEQ ID NO: 49 - DNA, 34 bp, Artificial Sequence
  Description of Artificial Sequence Synthetic primer
ccgctcgagctatgctatgtttggataagaagac

SEQ ID NO: 50 - DNA, 24 bp, Artificial Sequence
  Description of Artificial Sequence Synthetic primer
gcatccttcaagtatcaaaacctg

SEQ ID NO: 51 - DNA, 20 bp, Artificial Sequence
  Description of Artificial Sequence Synthetic primer
tcatgcaactgggtaggaag

SEQ ID NO: 52 - DNA, 22 bp, Artificial Sequence
  Description of Artificial Sequence Synthetic primer
tccaccttcaagtaccaaaacg

SEQ ID NO: 53 - DNA, 23 bp, Artificial Sequence
  Description of Artificial Sequence Synthetic primer
tcacattgcaactggataggaag

SEQ ID NO: 54 - DNA, 22 bp, Artificial Sequence
  Description of Artificial Sequence Synthetic primer
aaaggaaatcacaagcttactg

SEQ ID NO: 55 - DNA, 23 bp, Artificial Sequence
  Description of Artificial Sequence Synthetic primer
ctaagccacaactgggtatgatg

SEQ ID NO: 56 - DNA, 21 bp, Artificial Sequence
  Description of Artificial Sequence Synthetic primer
gatcgttatctgccacgtgtc

SEQ ID NO: 57 - DNA, 24 bp, Artificial Sequence
  Description of Artificial Sequence Synthetic primer
ttaagcactgctacttccaccttg

SEQ ID NO: 58 - DNA, 20 bp, Artificial Sequence
  Description of Artificial Sequence Synthetic primer
tctgttcaatcgcccggtac

SEQ ID NO: 59 - DNA, 26 bp, Artificial Sequence
  Description of Artificial Sequence Synthetic primer
ttatacaaaccaagcatcaatgaagg

SEQ ID NO: 60 - DNA, 23 bp, Artificial Sequence
  Description of Artificial Sequence Synthetic primer
caaaaggctcctatcctccctac

SEQ ID NO: 61 - DNA, 24 bp, Artificial Sequence
  Description of Artificial Sequence Synthetic primer
ctagcttgaggttgtatggatagc

SEQ ID NO: 62 - DNA, 24 bp, Artificial Sequence
  Description of Artificial Sequence Synthetic primer
cctgttaaaactcatgacaaatcc

SEQ ID NO: 63 - DNA, 22 bp, Artificial Sequence
  Description of Artificial Sequence Synthetic primer
tcaagctgaagcatcctcaaac

SEQ ID NO: 64 - protein, 4 aa, Unknown
  Description of Unknown “KDEL” motif peptide
KDEL